(19) United States
(12) Patent Application Publication
(10) Pub. No.: US 2023/0405125 A1
(43) Pub. Date: Dec. 21, 2023

(54) TREATMENT OF RECURRENT GIOBLASTOMA WITH PERILLYL ALCOHOL

(71) Applicant: NeOnc Technologies, Inc., Los Angeles, CA (US)

(72) Inventor: Thomas C. CHEN, La Canada, CA (US)

(21) Appl. No.: 18/251,839

(22) PCT Filed: Nov. 12, 2021
(86) PCT No.: PCT/US2021/059117

§ 371 Date: May 4, 2023

Related U.S. Application Data
(60) Provisional application No. 63/112,799, filed on Nov. 12, 2020.

(51) Int. Cl. A61K 47/10; A61P 35/00; A61K 45/06

(52) U.S. Cl. CPC A61K 47/10; A61P 35/00; A61K 45/06

(57) ABSTRACT

The present invention provides an intranasal glioblastoma therapy with purified perillyl alcohol. Patients with recurrent glioblastoma when treated with perillyl alcohol purified by the disclosed methods showed improved survival when compared to historical controls. Glioblastoma patients with an isocitrate dehydrogenase 1 (IDH1)-mutation showed improved survival when compared with wild-type IDH patients.

### CROSS-REFERENCE TO RELATED APPLICATIONS

The application claims priority to U.S. Provisional Application No. 63/112,799 filed Nov. 12, 2020, the disclosure of which is incorporated by reference herein in its entirety.

### FIELD OF THE INVENTION

The present invention relates to monoterpene or sesquiterpene compositions. In particular, the present invention relates to using monoterpenes (such as (S)-perillyl alcohol) or sesquiterpenes having a purity greater than about 98.5% (w/w) to treat nervous system tumors.

### BACKGROUND OF THE INVENTION

Malignant gliomas, the most common form of central nervous system (CNS) cancers, are currently considered essentially incurable. Among the various malignant gliomas, anaplastic astrocytomas (Grade III) and glioblastoma multiforme (GBM; Grade IV) have an especially poor prognosis due to their aggressive growth and resistance to currently available therapies. The present standard of care for malignant gliomas consists of surgery, ionizing radiation, and chemotherapy. Despite recent advances in medicine, the past 50 years have not seen any significant improvement in prognosis for malignant gliomas. Wen et al. Malignant gliomas in adults. New England J Med. 359: 492-507, 2008. Stupp et al. Radiotherapy plus concomitant and adjuvant temozolomide for glioblastoma. New England J Med. 352: 987-996, 2005.

A major reason for the poor prognosis of malignant gliomas is the difficulty in delivering a sufficient quantity of chemotherapeutic agents to the brain. Drug access to the brain is limited by the blood brain barrier (BBB). The concentration of drugs that finally reach the brain is further decreased by hepatic first-pass metabolism and urinary excretion. Therefore, invasive surgeries are often required, such as tumor resection, stereotactic injection of anti-tumor medication, or placement of catheters for convection enhanced delivery of medication.

Intranasal delivery of a drug offers a novel non-invasive therapy to bypass the blood brain barrier and to rapidly deliver pharmaceutical agents to the CNS directly. Intranasally administered drugs reach the parenchymal tissues of the brain, spinal cord and/or cerebrospinal fluid (CSF) within minutes. In addition to delivery via the olfactory tract and trigeminal nerves, it appears from animal studies that the therapeutic drug is also delivered systemically through the nasal vasculature. Hashizume et al. New therapeutic approach for brain tumors: intranasal delivery of telomerase inhibitor GRN163. Neuro-oncology 10: 112-120, 2008. Thorne et al. Delivery of insulin-like growth factor-1 to the rat brain and spinal cord along olfactory and trigeminal pathways following intranasal administration. Neuroscience 127: 481-496, 2004. Intranasal delivery of therapeutic agents may provide a systemic method for treating other types of cancers, such as lung cancer, prostate cancer, breast cancer, hematopoietic cancer and ovarian cancer, etc.

Perillyl alcohol (POH), a naturally occurring monoterpene, has been suggested to be an effective agent against a variety of cancers, including CNS cancer, breast cancer, pancreatic cancer, lung cancer, melanomas and colon cancer. Gould, M. Cancer chemoprevention and therapy by monoterpenes. Environ Health Perspect. 1997 June; 105 (Suppl 4): 977-979. Oral perillyl alcohol has been used in a recent phase I trial sponsored by the National Cancer Institute. Although oral perillyl alcohol did not induce severe adverse effects, it was generally poorly tolerated, mainly due to gastrointestinal side effects. In addition, its anti-cancer efficacy was limited. As a result, the use of oral perillyl alcohol was discontinued. Ripple et al. Phase I clinical and pharmacokinetic study of perillyl alcohol administered four times a day. Clinical Cancer Res 6: 390-6, 2000.

In order to minimize the gastrointestinal side effects of oral POH and to provide a means of delivering POH directly to the central nervous system, a nasal formulation of POH (see below) for direct intranasal delivery of POH to malignant brain tumors was studied by Dr. Clovis Fonseca at the Fluminese University in Brazil. Da Fonseca, et al. Anaplastic oligodendroglioma responding favorably to intranasal delivery of perillyl alcohol: a case report and literature review, Surgical Neurology (2006) 66:611-615. This formulation of commercial grade POH combined with a solvent cocktail, has already been delivered to 150 patients with recurrent malignant gliomas, with minimal side effect and a six month 50% progression free survival rate. Da Fonseca et al. Correlation of tumor topography and peritumoral edema of recurrent malignant gliomas with therapeutic response to intranasal administration of perillyl alcohol. Invest New Drugs 2009, Jan. 13.

Commercial grade perillyl alcohol, with purities ranging from 85% to 96%, is typically purified from natural products, or by synthetically modifying natural products such as beta-pinene (extracted from pine trees). Inevitably, perillyl alcohol obtained through these routes is contaminated by its isomers and other impurities which have similar physicochemical properties, and, therefore, are extremely difficult to remove from perillyl alcohol by conventional purification methods such as fractional distillation or chromatography. Isomers of perillyl alcohol and other impurities may be potentially inhibitory towards the desired therapeutic properties of perillyl alcohol.

Consequently, there is still a need to prepare highly purified perillyl alcohol and use this material in the treatment of CNS cancers such as malignant gliomas, as well as other aggressive brain tumors. Purified perilly alcohol may be administered alone or in combination with other treatment methods including radiation, standard chemotherapy, and surgery. The administration can also be through various routes including intranasal, oral, oral-tracheal for pulmonary delivery, and transdermal.

### SUMMARY OF THE INVENTION

The present invention provides for a process of purifying (S)-perillyl alcohol comprising the steps of: (a) derivatizing a mixture comprising (S)-perillyl alcohol to form a perillyl alcohol derivative, wherein the perillyl alcohol derivative has at least one property that allows it to be separated from the mixture; (b) separating the perillyl alcohol derivative from the mixture using the property for separation; (c) releasing the (S)-perillyl alcohol from the perillyl alcohol derivative from step (b); and, (d) isolating the (S)-perillyl alcohol from step (c). The (S)-perillyl alcohol has a purity greater than about 98.5% (w/w), greater than about 99.0% (w/w), or greater than about 99.5% (w/w). In certain embodiments, the mixture further comprises natural-product-derived or other impurities. The property of the perillyl alcohol derivative can be to form crystals, and the separation in step (b) can, therefore, be through crystallization. The separation in step (b) may also be through chromatography. The perillyl alcohol derivative can be a perillyl alcohol ester. In one embodiment, the perillyl alcohol ester is a benzoate ester, such as 3,5-dinitrobenzoate ester.

The invention also encompasses an (S)-perillyl alcohol, where the (S)-perillyl alcohol has a purity greater than about 98.5% (w/w), greater than about 99.0% (w/w), or greater than about 99.5% (w/w).

The invention further provides for a pharmaceutical composition comprising (S)-perillyl alcohol having a purity greater than about 98.5% (w/w). The (S)-perillyl alcohol may have a purity greater than about 98.5% (w/w). The pharmaceutical composition may contain from about 0.1% (w/w) to about 100% (w/w) (S)-perillyl alcohol. In addition, the pharmaceutical composition may comprise a chemotherapeutic agent, as well as at least one pharmaceutically acceptable excipient. The chemotherapeutic agent may be a DNA alkylating agent, a topoisomerase inhibitor, an endoplasmic reticulum stress inducing agent, a platinum compound, an antimetabolite, an enzyme inhibitor, a receptor antagonist, a therapeutic antibody, or a vaccine. In certain embodiments, the chemotherapeutic agent is dimethyl-celecoxib (DMC), irinotecan (CPT-11), temozolomide, or rolipram. The pharmaceutical composition can be administered alone, or may be administered before, during or after radiation, or before, during or after the administration of a chemotherapeutic agent. The routes of administration include inhalation, intranasal, oral, intravenous, subcutaneous and intramuscular injection. The pharmaceutical composition can be administered intranasally by an intranasal spray device, an atomizer, a nebulizer, a metered dose inhaler (MDI), a pressurized dose inhaler, an insufflator, an intranasal inhaler, a nasal spray bottle, a unit dose container, a pump, a dropper, a squeeze bottle, or a bi-directional device. The pharmaceutical composition may be administered intranasally in the form of a gel, an ointment, a nasal emulsion, a lotion, a cream, a nasal tampon, or a bioadhesive strip.

The present invention further provides for a method of treating cancer, comprising the step of delivering to a mammal a therapeutically effective amount of (S)-perillyl alcohol having a purity greater than about 98.5% (w/w). The (S)-perillyl alcohol may be admixed or coformulated with a therapeutic agent, for example, a chemotherapeutic agent. The cancer may be a tumor of the nervous system, such as a glioblastoma, or other tumors.

The present invention provides for an article of manufacture (e.g., a kit) comprising (S)-perillyl alcohol formulated for intranasal administration, and a device for intranasal administration of the (S)-perillyl alcohol, wherein the (S)-perillyl alcohol has a purity of greater than about 98.5% (w/w). The device may be an intranasal spray device, an atomizer, a nebulizer, a metered dose inhaler (MDI), a pressurized dose inhaler, an insufflator, an intranasal inhaler, a nasal spray bottle, a unit dose container, a pump, a dropper, a squeeze bottle, or a bi-directional device. The article of manufacture may further comprise printed matter which states the (S)-perillyl alcohol is to be used to treat cancer, such as glioblastoma. The printed matter may further state the (S)-perillyl alcohol is to be administered alone, or administered in combination with radiation, surgery or chemotherapeutic agents.

Also provided for is a method of inhibiting the growth of a cell, comprising the step of contacting the cell with an effective amount of (S)-perillyl alcohol having a purity greater than about 98.5% (w/w). The contacting may occur in vitro or in vivo. The cell may be a glioma cell, a meningioma cell, a pituitary adenoma cell, a lung cancer cell, a prostate cancer cell, a breast cancer cell, a hematopoietic cancer cell, a melanoma cell, or an ovarian cancer cell. The cell may be a temozolomide-resistant cell or a cancer stem cell.

The compositions and methods of the present invention may be used to decrease or inhibit angiogenesis. The present compositions and methods may decrease or inhibit production of pro-angiogenic cytokines, including, but not limited to, vascular endothelial growth factor (VEGF) and interleukin 8 (IL8).

The compositions and methods of the present invention may be used to increase paracellular permeability, for example, paracellular permeability of endothelial cells or epithelial cells. The present compositions and methods may be used to increase blood brain barrier permeability.

The methods also comprise treating a tumor of the nervous system in a patient, where the patient has a mutated isocitrate dehydrogenase 1 (IDH1) gene, the method comprising administering to the patient a pharmaceutical composition comprising perillyl alcohol (POH) purified according the methods the invention, or administration of a perillyl alcohol carbamate, where the perillyl alcohol carbamate is perillyl alcohol covalently bound via a carbamate linking group to a therapeutic agent such as temozolamide, rolipram or dimethyl celecoxib. The tumor of the central nervous system can be a glioblastoma or a recurrent glioblastoma. The treatment can be combined with another therapeutic agent, such as a chemotherapeutic agent.

### BRIEF DESCRIPTION OF THE DRAWINGS

FIG. 1: Progression-free survival of different cohorts.

Shown is progression-free survival of patients within the first 6 months (PFS-6) after initiation of NEO100 treatment, separated into the four cohorts with n=3 patients each.

FIGS. 2A-2B: Examples of radiographic responses.

(FIG. 2A) MRI scans of Patient 202 before treatment and after 10 months of NEO100 show partial response. (FIG. 2B) MRI scans of Patient 301 before and after 12 months of NEO100, showing lack of recurrence during NEO100 treatment.

FIGS. 3A-3C. OVERALL survival. (FIG. 3A) Shown is the Survival of all Patients (n=12) within the first 24 months after initiation of NEO100 treatment, irrespective of the number of treatment cycles that were completed. Overall survival at 12 months (OS-12) and 24 months (OS-24) is indicated. Median OS is shown at 15 months. Note that one patient (ID 401) was censored at 4 months (tick mark) because lie was lost to follow-up. (FIG. 31B) Shown is the survival of patients within the first 24 months after initiation of NEO100 treatment, separated into groups of patients who completed at least 6 cycles (n=4; indicated as >5 cycles) and those who completed fewer than 5 cycles (n=7; <5 cycles). The status of Patient 401 was lost to follow-up after completion of 4 cycles and progressive disease, and therefore he was not included in this comparison. (FIG. 3C) Shown is the survival of patients within the first 24 months after initiation of NEO100 treatment, separated as per IDH1 status in their tumor tissues. Four of 5 patients (80%) with mutated IDH1 survived at least 24 months. Six patients with wild type IDH1 had succumbed to their disease by 18 months. P=0.018 (log-rank test). Patient 401 (IDH1 wild type (“WT or wt”)) was censored at s (tick mark).

FIG. 4: Perillic acid concentrations in patient plasma.

Concentration of perillic acid was determined in plasma from all patients (patient ID #shown in squares in each graph). Blood was drawn at different time points after completion of NEO100 inhalation on the first day of Cycle 1, the eighth day of Cycle 1, and the first day of Cycle 2. Boxes to the right show C-max averages for each cohort for each of these three measurements. Each average was derived from 3 patients, except for one missing set of data from patient 401.

FIG. 5: Overall survival after NEO100 Administration—Months since starting NEO100 treatment.

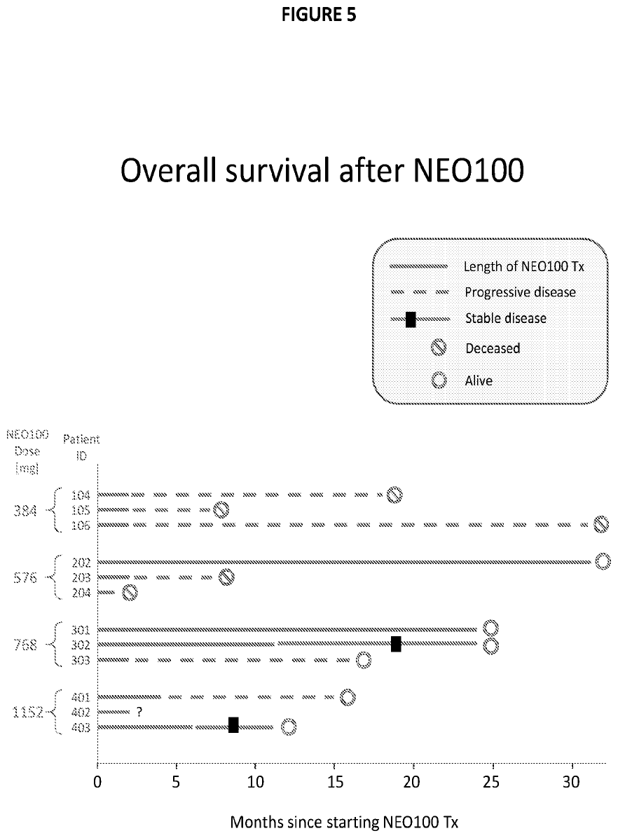

### DETAILED DESCRIPTION OF THE INVENTION

Abbreviations: cGMP: current good manufacturing practice; CNS: central nervous system; GBM: glioblastoma (formerly: glioblastoma multiforme); IDH1: isocitrate dehydrogenase 1; OS: overall survival; MGMT: 06-methylguanine-DNA methyltransferase; PFS: progression-free survival; WHO: World Health Organization; NEO100—perillyl alcohol purified according the methods and materials described herein and in U.S. Pat. Nos. 8,50,773, 9,133,085, 9,480,659, 9,498,448, 9,700,524 and 10,4757,618 which are incorporated in their entirety by reference: NEO100 is (S)-perillyl alcohol purified by the methods set forth above and has a purity greater than about greater than about 99.0% (w/w); IDH1: Isocitrate dehydrogenase 1: POH is referred to as perillyl alcohol and is also referred to as p-metha 1,7-diene-6-ol; the U.S. patents describing the perillyl alcohol conjugates are U.S. Pat. Nos. 8,916,545, 9,499,461, 9,580,372, 9,663,428 and 10,092,562, which are incorporated in their entirety by reference; IDH1; mut.—mutant; WT or w.t. is wild type.

The present invention provides for methods of purifying perillyl alcohol from its isomers (including enantiomer) and other impurities that typically accompany perillyl alcohol when it is produced from natural products and/or synthetic sources. Perillyl alcohol may be purified by derivatizing perillyl alcohol to produce a crystalline derivative such as its 3,5-dinitrobenzoate ester. The perillyl alcohol derivative can then be separated from its accompanying contaminants (whether or not the contaminants are also present as derivatives or not) by suitable techniques, such as conventional crystallization, or preparative chromatography. The purified perillyl alcohol derivative can then be converted to perillyl alcohol which has a purity greater than about 98.5% (w/w). The purified perillyl alcohol may be administered to a subject alone, or may be co-administered together with other agents. For example, the purified perillyl alcohol may be used to sensitize a cancer patient to radiation or chemotherapy. Compared to commercially available (S)-perillyl alcohol, the purified (S)-perillyl alcohol demonstrates disproportionately enhanced activity in cellular assays and other therapeutic test models.

The present invention provides for a process of preparing a purified form of a monoterpene or sesquiterpene or a monoterpene derivative. The monoterpene (or sesquiterpene) is purified by the following steps: (a) derivatizing a mixture comprising monoterpene (or sesquiterpene) to form a monoterpene (or sesquiterpene) derivative, wherein the monoterpene (or sesquiterpene) derivative has at least one property that allows it to be separated from the mixture; (b) separating the monoterpene (or sesquiterpene) derivative from the mixture using the property for separation; (c) releasing the monoterpene (or sesquiterpene) from the monoterpene (or sesquiterpene) derivative from step (b); and, (d) isolating the monoterpene (or sesquiterpene) from step (c). The purified monoterpene (or sesquiterpene) may have a purity greater than about 98.5% (w/w), about 99.0% (w/w), or about 99.5% (w/w). In certain embodiments, the mixture further comprises natural-product-derived or other impurities. The (S)-perillyl alcohol has a purity greater than about 98.5% (w/w), greater than about 99.0% (w/w), or greater than about 99.5% (w/w).

The property of the monoterpene (or sesquiterpene) can be to form crystals, and the separation in step (b) can, therefore, be through crystallization. The monoterpene (or sesquiterpene) is purified by the following steps: (a) derivatizing the monoterpene (or sesquiterpene) to form a monoterpene (or sesquiterpene) derivative; (b) crystallizing the monoterpene (or sesquiterpene) derivative; (c) separating the monoterpene (or sesquiterpene) derivative crystals of step (b); (d) converting the separated monoterpene (or sesquiterpene) derivative to monoterpene (or sesquiterpene); and (e) isolating the monoterpene (or sesquiterpene).

The separation of the monoterpene (or sesquiterpene) from the mixture may also be through other suitable separation techniques known in the art, including, but not limited to, chromatography, adsorption, centrifugation, decantation, distillation, electrophoresis, evaporation, extraction, flotation, filtration, precipitation, sedimentation. Wikipedia—Separation Process. Retrieved on Feb. 11, 2010 from URL: http://en.wikipedia.org/wiki/Separation_of_mixtures. The property of the monoterpene (or sesquiterpene) derivative useful for separation of the derivative from the mixture can be any of its physicochemical properties that are different from that of the other components in the mixture. The physicochemical properties include, but are not limited to, solubility, polarity, partition coefficient, affinity, size, hydrodynamic diameter, and charge. The monoterpene (or sesquiterpene) derivative can be prepared where the derivative has at least one different property than that of its isomers, structural variants, or contaminants present in the starting material. The chromatography can be any suitable preparative chromatography, including, but not limited to, gas chromatography (GC), high pressure liquid chromatography (HPLC), affinity chromatography, ion exchange chromatography, size exclusion chromatography, and reversed-phase chromatography.

In one embodiment, the monoterpene may be (S)-perillyl alcohol, and the derivatization reaction can involve esterification. For example, (S)-perillyl alcohol may be prepared using a 3,5-dinitrobenzoate ester derivative.

The present invention further provides for a monoterpene (or sesquiterpene) composition having a purity of greater than about 98.5% (w/w), greater than about 99.0% (w/w), or greater than about 99.5% (w/w).

The purified monoterpene (or sesquiterpene) may be formulated into a pharmaceutical composition, where the monoterpene (or sesquiterpene) is present in amounts ranging from about 0.01% (w/w) to about 100% (w/w), from about 0.1% (w/w) to about 80% (w/w), from about 1% (w/w) to about 70% (w/w), from about 10% (w/w) to about 60% (w/w), or from about 0.1% (w/w) to about 20% (w/w). In addition, the pharmaceutical composition may contain a therapeutic agent, such as a chemotherapeutic agent. The therapeutic agent may be dissolved in perillyl alcohol. The present compositions can be administered alone, or may be co-administered together with radiation or another agent (e.g., a chemotherapeutic agent), to treat a disease such as cancer. Treatments may be sequential, with the monoterpene (or sesquiterpene) being administered before or after the administration of other agents. Alternatively, agents may be administered concurrently. The route of administration may vary, and can include, inhalation, intranasal, oral, transdermal, intravenous, subcutaneous or intramuscular injection.

The present invention also provides for a method of treating a disease such as cancer, comprising the step of delivering to a patient a therapeutically effective amount of a purified monoterpene (or sesquiterpene) prepared by the methods of the present invention.

The compositions of the present invention may contain one or more types of monoterpene (or sesquiterpene). Monoterpenes include terpenes that consist of two isoprene units and have the molecular formula C10H16. Monoterpenes may be linear (acyclic) or contain rings. Monoterpenoids, produced by biochemical modifications such as oxidation or rearrangement of monoterpenes, and pharmaceutically acceptable salts of monoterpenes or monoterpenoids, are also encompassed by the present invention. Examples of monoterpenes and monoterpenoids include, perillyl alcohol (S(−)) and R(+)), geranyl pyrophosphate, ocimene, myrcene, geraniol, citral, citronellol, citronellal, linalool, pinene, terpineol, terpinen, limonene, terpinenes, phellandrenes, terpinolene, terpinen-4-ol (or tea tree oil), pinene, terpineol, terpinen; the terpenoids such as p-cymene which is derived from monocyclic terpenes such as menthol, thymol and carvocrol; bicyclic monoterpenoids such as camphor, borneol and eucalyptol.

Monoterpenes may be distinguished by the structure of a carbon skeleton and may be grouped into acyclic monoterpenes (e.g., myrcene, (Z)- and (E)-ocimene, linalool, geraniol, nerol, citronellol, myrcenol, geranial, citral a, neral, citral b, citronellal, etc.), monocyclic monoterpenes (e.g., limonene, terpinene, phellandrene, terpinolene, menthol, carveol, etc.), bicyclic monoterpenes (e.g., pinene, myrtenol, myrtenal, verbanol, verbanon, pinocarveol, carene, sabinene, camphene, thujene, etc.) and tricyclic monoterpenes (e.g. tricyclene). See Encyclopedia of Chemical Technology, Fourth Edition, Volume 23, page 834-835.

Sesquiterpenes of the present invention include terpenes that consist of three isoprene units and have the molecular formula C15H24. Sesquiterpenes may be linear (acyclic) or contain rings. Sesquiterpenoids, produced by biochemical modifications such as oxidation or rearrangement of sesquiterpenes, are also encompassed by the present invention. Examples of sesquiterpenes include farnesol, farnesal, farnesylic acid and nerolidol.

The purified monoterpene (or sesquiterpene) is prepared using the derivatized monoterpene (or sesquiterpene), which may be separated from its accompanying contaminants (such as its isomers) by crystallization. The crystallization and purification may also enhance the chiral purity of the monoterpene (or sesquiterpene).

The derivatives of monoterpene (or sesquiterpene) include, but are not limited to, esters, alcohols, aldehydes and ketones of the monoterpene (or sesquiterpene). Monoterpene (or sesquiterpene) alcohols may be derivatized to esters, aldehydes or acids. The derivatives of monoterpene (or sesquiterpene) can be used to regenerate the monoterpene (or sesquiterpene) through chemical reactions known to a person skilled in the art. For example, an ester of a monoterpene (or sesquiterpene) can be hydrolyzed to generate the monoterpene (or sesquiterpene).

In one embodiment, a monoterpene (or sesquiterpene) is purified using an ester of the monoterpene (or sesquiterpene). The purification process includes the following steps: (a) derivatizing a monoterpene (or sesquiterpene) to produce an ester of the monoterpene (or sesquiterpene); (b) crystallizing the ester of the monoterpene (or sesquiterpene); (c) separating crystals of the ester of the monoterpene (or sesquiterpene) of step (b); (d) converting the ester of the monoterpene (or sesquiterpene) to the monoterpene (or sesquiterpene); and (e) isolating the monoterpene (or sesquiterpene).

Esters of the monoterpene (or sesquiterpene) alcohols of the present invention can be derived from an inorganic acid or an organic acid. Inorganic acids include, but are not limited to, phosphoric acid, sulfuric acid, and nitric acid. Organic acids include, but are not limited to, carboxylic acid such as benzoic acid, fatty acid, acetic acid and propionic acid. Examples of esters of monoterpene (or sesquiterpene) alcohols include, but are not limited to, carboxylic acid esters (such as benzoate esters, fatty acid esters (e.g., palmitate ester and linoleate ester), acetates, propionates (or propanoates), and formates), phosphates, sulfates, and carbamates (e.g., N,N-dimethylaminocarbonyl). Wikipedia—Ester. Retrieved from URL: http://en.wikipedia.org/wiki/Ester, Nov. 11, 2021).

In one embodiment, the derivatives are benzoate esters including, but not limited to, 3,5-dinitrobenzoate ester, 4-nitrobenzoate ester, 3-nitrobenzoate ester, 4-chlorobenzoate ester, 3,4,5-trimethoxybenzoate ester and 4-methoxybenzoate ester, esters of hydroxybenzoic acid such as the methyl, ethyl, propyl, isopropyl, butyl, isobutyl, hexyl, heptyl and benzyl esters. (See, for example, Wikipedia—Benzoate ester http://commons.wikimedia.org/wiki/Category:Benzoate_esters).

A specific example of a monoterpene that may be used in the present invention is perillyl alcohol (commonly abbreviated as POH). Perillyl alcohol compositions of the present invention can contain (S)-perillyl alcohol, (R)-perillyl alcohol, or a mixture of (S)-perillyl alcohol and (R)-perillyl alcohol.

Perillyl alcohol may be purified by the following steps: (a) derivatizing a mixture comprising perillyl alcohol to form a perillyl alcohol derivative, wherein the perillyl alcohol derivative has at least one property that allows it to be separated from the mixture; (b) separating the perillyl alcohol derivative from the mixture using the property for separation; (c) releasing the perillyl alcohol from the perillyl alcohol derivative from step (b); and (d) isolating the perillyl alcohol from step (c). In certain embodiments, the mixture further comprises natural-product-derived or other impurities.

Perillyl alcohol may be purified using the methods of the present invention with perillyl alcohol derivatives. The derivatives include, perillyl alcohol esters, dihydroperillic acid, and perillic acid. The derivatives of perillyl alcohol may also include its oxidative and nucleophilic/electrophilic addition derivatives. U.S. Patent Publication No. 20090031455. U.S. Pat. Nos. 6,133,324 and 3,957,856. Many examples of derivatives of perillyl alcohol are reported in the chemistry literature (CAS Scifinder search output file, retrieved Jan. 25, 2010).

In a specific example, perillyl alcohol is purified by: (a) derivatizing perillyl alcohol to produce a perillyl alcohol ester; (b) crystallizing the perillyl alcohol ester; (c) separating the perillyl alcohol ester crystals of step (b) (e.g., from a mother liquor); (d) converting the separated perillyl alcohol ester to generate perillyl alcohol; and (e) isolating the perillyl alcohol. The derivative of perillyl alcohol can be used to regenerate the perillyl alcohol through chemical reactions known to a person skilled in the art. For example, an ester of perillyl alcohol, such as a 3,5-dinitrobenzoate ester, can be hydrolyzed to generate perillyl alcohol.

In certain embodiments, esters or ethers of perillyl alcohol may be prepared by reacting perillyl alcohol with acid chlorides or alkyl chlorides, the chemical structures of which are shown below.

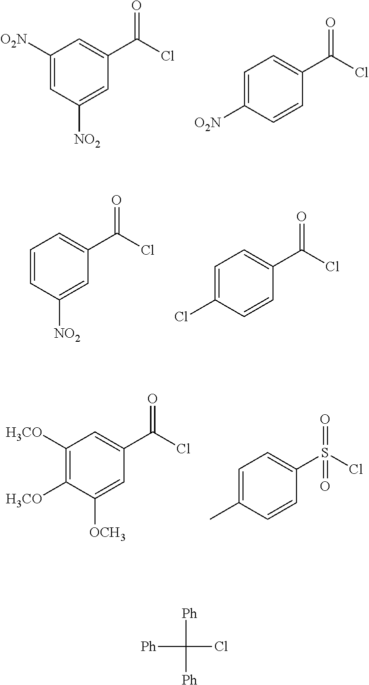

For the esterification reaction, the molar ratio of perillyl alcohol to acid chloride (or alkyl chloride) may range from about 1:1 to about 1:2, from about 1:1 to about 1:1.5, including, for example, about 1:1.05, about 1:1.1, about 1:1.2, about 1:1.3, or about 1:1.4. Suitable reaction solvents include, but are not limited to, dichloromethane, diethyl ether, diisopropyl ether, and methyl-t-butyl ether. The reaction may be performed at a temperature ranging from about −5.degree. C. to about 50.degree. C., or from about −5.degree. C. to 25.degree. C. Suitable bases that may be included in the reaction include, but are not limited to, organic bases, such as triethylamine, di-isopropylamine, N,N′-diisopropylethylamine, butylamine, sodium methoxide, potassium methoxide, and potassium-t-butoxide. The esters thus generated are 3,5-dinitrobenzoate ester, 4-nitrobenzoate ester, 3-nitrobenzoate ester, 4-chlorobenzoate ester, 3,4,5-trimethoxybenzoate ester, 4-methoxybenzoate ester and triphenylmethyl ester. The details of the chemical reactions are described in the Examples below.

The crystallizable monoterpene (or sesquiterpene) derivative may be purified by crystallization or preparative chromatography. Crystallization separates a product from a liquid feedstream, often in extremely pure form, by cooling the feedstream or adding precipitants which lower the solubility of the desired product so that it forms crystals. For crystallization to occur, the solution must be supersaturated. This means that the solution has to contain more dissolved solute entities than it would contain under the equilibrium (saturated solution). This can be achieved by various methods, such as 1) solution cooling; 2) addition of a second solvent to reduce the solubility of the solute (a technique known as antisolvent or drown-out); 3) chemical reaction; and 4) change in pH. Solvent evaporation, spherical crystallization, fractional crystallization, fractional freezing procedures, and other suitable methods can also be used. Mersmann, A. Crystallization Technology Handbook. Edition 2 (2001), published by CRC Press. Myerson et al. Crystallization As a Separations Process (ACS Symposium Series) (1990), published by American Chemical Society.

In a specific example, crystallization of 3,5-dinitrobenzoate ester of perillyl alcohol is carried out as follows. The aqueous layer containing 3,5-dinitrobenzoate ester is extracted with dichloromethane and washed with water. The organic layer which contains the 3,5-dinitrobenzoate ester is dried over sodium sulphate. The organic layer is then filtered and concentrated. The resulting residue is finally crystallized from a diisopropyl ether mother liquor. A mother liquor is the part of a liquid that is above the crystal solids, and, thus, can be separated from the crystals. The separation of the crystals from the mother liquor can be carried out using any suitable techniques, including, but not limited to, filtration (with or without the assistance of pressure and/or vacuum), centrifugation, and decantation.

Suitable solvents for crystallization of benzoate ester of perillyl alcohol include, but are not limited to, ketone solvents (such as acetone, methyl ethyl ketone, methyl isobutyl ketone, n-butanone, and t-butylketone); nitrile solvents (such as acetonitrile, and propionitrile); halogenated solvents (such as dichloromethane 1,2-dichloroethane, and chloroform); esters (such as ethyl acetate, n-propylacetate, isopropyl acetate, and t-butylacetate); ethers (such as diethyl ether, diisopropyl ether, methyl-t-butyl ether, tetrahydrofuran and 1,4-dioxane); hydrocarbon solvents (such as hexanes, cyclohexane, toluene and xylene); and mixtures thereof. In one embodiment, the solvent contains methyl-t-butyl ether. The dissolution of benzoate ester in methyl-t-butyl ether (7-10 volumes) may be performed at an elevated temperature, if required, to achieve the desired concentration. Further, an activated charcoal treatment may be performed to remove colored impurities or to reduce the content of heavy metals, if any, or to remove any extraneous matter from the solution containing benzoate ester. The crystallization from the resultant reaction mixture may be carried out by cooling the reaction mixture to a lower temperature of about 25° C. to about 0° C. Separation of the crystals may be carried out by removal of the solvent followed by cooling the reaction mixture. Solvent may be removed by suitable techniques including evaporation using a rotary evaporator, such as a Buchi rotavapor under vacuum. Crystals may be isolated from the reaction mixture by any conventional technique such as filtration by gravity or by suction. In one embodiment, the benzoate ester may be isolated by filtration and, if desired, may be further washed with a solvent. The benzoate ester may be dried by any of the conventional techniques such as drying in a tray dryer, vacuum dryer, or air oven. The drying may be carried out at a temperature of about 30° C. to about 60° C. in a vacuum oven.

The purity of the crystallizable monoterpene (or sesquiterpene) derivative, and therefore, the purity of the monoterpene (or sesquiterpene), may be further improved by recrystallization. Various techniques can be used, such as single-solvent recrystallization, multi-solvent recrystallization, hot filtration-recrystallization, as well as other suitable recrystallization techniques which are well known in the art. Wikipedia—Recrystallization. Retrieved from URL: http://en.wikipedia.org/wiki/Recrystallization (chemistry).

For example, U.S. Pat. No. Re. 32,241 describes an apparatus having a component crystallize on a cooled surface as material containing the component flows down. U.S. Pat. No. 4,666,456 describes continuous partial crystallization of a compound from a liquid mixture in which the mixture is fed through a cascade of cooling sections. U.S. Pat. No. 5,127,921 provides a multi-stage recrystallization procedure including controlling reflux ratio conditions by regulating quantities of crystals and mother liquor reflux materials.

The purity of the perillyl alcohol made by the above described processes may be greater than about 98.5% (w/w), greater than about 99% (w/w), greater than about 99.5% (w/w), or greater than about 99.9% (w/w).

In certain embodiments, the compounds of the invention contain one or more chiral centers. The term “purity” can also encompass chiral purity. The purity of a stereoisomer of a monoterpene (or sesquiterpene) refers to chemical purity and/or chiral purity of the stereoisomer. For example, the purity of (S)-perillyl alcohol can include both the chemical purity and the chiral purity of (S)-perillyl alcohol. The chiral purity of a stereoisomer of the monoterpene (or sesquiterpene) may be greater than about 98.5% (w/w), greater than about 99% (w/w), greater than about 99.5% (w/w), or greater than about 99.9% (w/w).

The chiral purity of (S)-perillyl alcohol may be greater than about 98.5% (w/w), greater than about 99% (w/w), greater than about 99.5% (w/w), or greater than about 99.9% (w/w). In certain embodiments, the specific optical rotation of (S)-perillyl alcohol of the present invention may range from −87.95.degree. to −91.9.degree, when the specific optical rotation is measured at 22° C. with the sample concentration at 1 g/ml in MeOH (see Table 1 for examples of specific optical rotation of (S)-perillyl alcohol).

TABLE 1
Estimated chiral purity of perillyl alcohol samples based on optical rotation

| | | | | | Purity by | Specific | |
|---|---|---|---|---|---|---|---|
| | | | | | Neone | optical | |
| | | | | Parity by | analysis | rotation | |
| | | | | Vendor | (%) GC | (C = 1, | |
| Vendor | Lot# | Quantity | | (%) | (by area) | MeOH) | Sample description |
| Wako | ASK0744 | 5.0 | g | 85 | 90.4 | −80.9° | Wako feed stock |
| Wako | KWH0744 | 400 | g | 85 | 89.5 | −81.5° | Wako feed stock |
| | (Neone | | | | | | |
| | Sample# 12) | | | | | | |
| Aldrich | MKAA4409 | 2 × 50 | g | 96 | 95.0 | −88.7° | Aldrich lab sample |
| Aldrich | MKAA0552 | 100 | g | * 90 | 96.2 | −87.6° | Aldrich bulk |
| | | | | | | | representative sample |
| Neone | SGP-527-130 | 1.0 | g | | 97.1 | −88.2° | Prepared from |
| | (Neone | | | | | | KWH0744 (single |
| | Sample# 07) | | | | | | crystallized from |
| | | | | | | | diisopropyl ether) |
| Neone | SGP-527-133 | 1.0 | g | | 98.7 | −87.9° | Prepared from |
| | (Neone | | | | | | KWH0744 |
| | Sample# 09) | | | | | | (Double recrystallized |
| | | | | | | | from diisopropyl ether |
| | | | | | | | then from 2-propanol) |
| Neone | SGP-527-138 | 1.0 | g | | 98.7 | −89.8° | Prepared from |
| | (Neone | | | | | | Aldrich |
| | Sample# 10) | | | | | | MKAA0552 |
| Neone | SGP-527-153 | 44.0 | g | | 98.6 | −91.9° | Prepared from |
| | (Neone | | | | | | Wako |
| | Sample# 13) | | | | | | KWH0744 |
| Neone | SGP-527-155 | 46.0 | g | | 98.6 | −91.7° | Prepared from |
| | (Neone | | | | | | Wako |
| | Sample# 14) | | | | | | KWH0744 |

The purity of the monoterpene (or sesquiterpene) may be assayed by gas chromatography (GC) or high pressure liquid chromatography (HPLC). Other techniques for assaying the purity of monoterpene (or sesquiterpene) and for determining the presence of impurities include, but are not limited to, nuclear magnetic resonance (NMR) spectroscopy, mass spectrometry (MS), GC-MS, infrared spectroscopy (IR), and thin layer chromatography (TLC). WHO Specifications and Evaluations for Public Health Pesticides: Malathion, World Health Organization, 2003. Chiral purity can be assessed by chiral GC or measurement of optical rotation.

Alternatively, the monoterpene (or sesquiterpene) may be purified by methods other than crystallizing the derivates. For example, a monoterpene (or sesquiterpene) derivative can be prepared where the derivative has different physicochemical properties (e.g., solubility or polarity) than that of its isomers, structural variants, or contaminants present in the starting material. Accordingly, the monoterpene (or sesquiterpene) derivative can be separated from the monoterpene (or sesquiterpene) by suitable separation techniques known in the art, such as preparative chromatography.

The purified monoterpene (or sesquiterpene) may be stable after storage. For example, after storage at about 5° C. for at least 3 months, the present composition may contain greater than about 98.5% (w/w), greater than about 99% (w/w), greater than about 99.5% (w/w), or greater than about 99.9% (w/w) monoterpene (or sesquiterpene). After storage at 25° C. and 60% relative humidity for at least 3 months, the present composition can contain greater than about 98.5% (w/w), greater than about 99% (w/w), greater than about 99.5% (w/w), or greater than about 99.9% (w/w) monoterpene (or sesquiterpene).

The invention also provides for methods of using monoterpenes (or sesquiterpenes) to treat a disease, such as cancer or other nervous system disorders. Monoterpenes (or sesquiterpenes) may be administered alone, or in combination with radiation, surgery or chemotherapeutic agents. The monoterpene or sesquiterpene may also be co-administered with antiviral agents, anti-inflammatory agents or antibiotics. The agents may be administered concurrently or sequentially. Monoterpenes (or sesquiterpenes) can be administered before, during or after the administration of the other active agent(s).

The monoterpenes (or sesquiterpenes) may also be used as a solvent or a permeation enhancer to deliver a therapeutic agent to the lesion site. For example, monoterpenes (or sesquiterpenes) may be used as a solvent or a permeation enhancer to deliver chemotherapeutic agents to tumor cells. The monoterpene or sesquiterpene may also be used as a solvent for vaccines, which may be delivered through any suitable route, such as intranasally.

The present invention also provides for using a derivative of monoterpene or sesquiterpene, such as a perillyl alcohol carbamate derivative. For example, the perillyl alcohol derivative may be a perillyl alcohol carbamate. The perillyl alcohol derivative may be perillyl alcohol conjugated with a therapeutic agent such as a chemotherapeutic agent. The monoterpene (or sesquiterpene) derivative may be formulated into a pharmaceutical composition, where the monoterpene (or sesquiterpene) derivative is present in amounts ranging from about 0.01% (w/w) to about 100% (w/w), from about 0.1% (w/w) to about 80% (w/w), from about 1% (w/w) to about 70% (w/w), from about 10% (w/w) to about 60% (w/w), or from about 0.1% (w/w) to about 20% (w/w). The present compositions can be administered alone, or may be co-administered together with radiation or another agent (e.g., a chemotherapeutic agent), to treat a disease such as cancer. Treatments may be sequential, with the monoterpene (or sesquiterpene) derivative being administered before or after the administration of other agents. For example, a perillyl alcohol carbamate may be used to sensitize a cancer patient to radiation or chemotherapy. Alternatively, agents may be administered concurrently. The route of administration may vary, and can include, inhalation, intranasal, oral, transdermal, intravenous, subcutaneous or intramuscular injection. The present invention also provides for a method of treating a disease such as cancer, comprising the step of delivering to a patient a therapeutically effective amount of a derivative of monoterpene (or sesquiterpene).

The derivatives of monoterpene (or sesquiterpene) include, but are not limited to, carbamates, esters, ethers, alcohols and aldehydes of the monoterpene (or sesquiterpene). Monoterpene (or sesquiterpene) alcohols may be derivatized to carbamates, esters, ethers, aldehydes or acids. Carbamate refers to a class of chemical compounds sharing the functional group

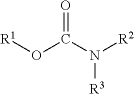

based on a carbonyl group flanked by an oxygen and a nitrogen. R¹, R² and R³ can be a group such as alkyl, aryl, etc., which can be substituted. The R groups on the nitrogen and the oxygen may form a ring. R¹—OH may be a monoterpene, e.g., POH. The R²—N—R³ moiety may be a therapeutic agent.

Carbamates may be synthesized by reacting isocyanate and alcohol, or by reacting chloroformate with amine. Carbamates may be synthesized by reactions making use of phosgene or phosgene equivalents. For example, carbamates may be synthesized by reacting phosgene gas, diphosgene or a solid phosgene precursor such as triphosgene with two amines or an amine and an alcohol. Carbamates (also known as urethanes) can also be made from reaction of a urea intermediate with an alcohol. Dimethyl carbonate and diphenyl carbonate are also used for making carbamates. Alternatively, carbamates may be synthesized through the reaction of alcohol and/or amine precursors with an ester-substituted diaryl carbonate, such as bismethylsalicylcarbonate (BMSC). U.S. Patent Publication No. 20100113819. Carbamates may be synthesized by the following approach:

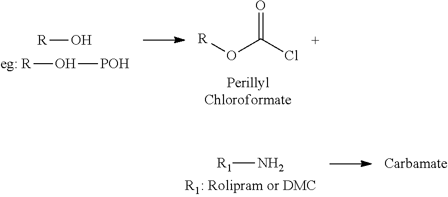

Suitable reaction solvents include, but are not limited to, tetrahydrofuran, dichloromethane, dichloroethane, acetone, and diisopropyl ether. The reaction may be performed at a temperature ranging from about −70° C. to about 80° C., or from about −65° C. to about 50° C. The molar ratio of perillyl chloroformate to the substrate R—NH₂ may range from about 1:1 to about 2:1, from about 1:1 to about 1.5:1, from about 2:1 to about 1:1, or from about 1.05:1 to about 1.1:1. Suitable bases include, but are not limited to, organic bases, such as triethylamine, potassium carbonate, N,N′-diisopropylethylamine, butyl lithium, and potassium-t-butoxide. Alternatively, carbamates may be synthesized by the following approach:

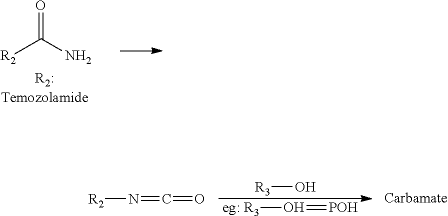

Suitable reaction solvents include, but are not limited to, dichloromethane, dichloroethane, toluene, diisopropyl ether, and tetrahydrofuran. The reaction may be performed at a temperature ranging from about 25° C. to about 110° C., or from about 30° C. to about 80° C., or about 50° C. The molar ratio of perillyl alcohol to the substrate R—N═C═O may range from about 1:1 to about 2:1, from about 1:1 to about 1.5:1, from about 2:1 to about 1:1, or from about 1.05:1 to about 1.1:1.

Esters of the monoterpene (or sesquiterpene) alcohols of the present invention can be derived from an inorganic acid or an organic acid. Inorganic acids include, but are not limited to, phosphoric acid, sulfuric acid, and nitric acid. Organic acids include, but are not limited to, carboxylic acid such as benzoic acid, fatty acid, acetic acid and propionic acid, and any therapeutic agent bearing at least one carboxylic acid functional group Examples of esters of monoterpene (or sesquiterpene) alcohols include, but are not limited to, carboxylic acid esters (such as benzoate esters, fatty acid esters (e.g., palmitate ester, linoleate ester, stearate ester, butyryl ester and oleate ester), acetates, propionates (or propanoates), and formates), phosphates, sulfates, and carbamates (e.g., N,N-dimethylaminocarbonyl). Wikipedia-Ester. Retrieved from URL: http://en.wikipedia.org/wiki/Ester.

A specific example of a monoterpene that may be used in the present invention is perillyl alcohol (commonly abbreviated as POH). The derivatives of perillyl alcohol include, perillyl alcohol carbamates, perillyl alcohol esters, perillic aldehydes, dihydroperillic acid, perillic acid, perillic aldehyde derivatives, dihydroperillic acid esters and perillic acid esters. The derivatives of perillyl alcohol may also include its oxidative and nucleophilic/electrophilic addition derivatives. U.S. Patent Publication No. 20090031455. U.S. Pat. Nos. 6,133,324 and 3,957,856. Many examples of derivatives of perillyl alcohol are reported in the chemistry literature (see Appendix A: CAS Scifinder search output file, retrieved Jan. 25, 2010).

In certain embodiments, a POH carbamate is synthesized by a process comprising the step of reacting a first reactant of perillyl chloroformate with a second reactant such as dimethyl celocoxib (DMC), temozolomide (TMZ) and rolipram. The reaction may be carried out in the presence of tetrahydrofuran and a base such as n-butyl lithium. Perillyl chloroformate may be made by reacting POH with phosgene. For example, POH conjugated with temozolomide through a carbamate bond may be synthesized by reacting temozolomide with oxalyl chloride followed by reaction with perillyl alcohol. The reaction may be carried out in the presence of 1,2-dichloroethane.

POH carbamates encompassed by the present invention include, but not limited to, 4-(bis-N,N′-4-isopropenyl cyclohex-1-enylmethyloxy carbonyl[5-(2,5-dimethyl phenyl)-3-trifluoromethylpyrazol-1-yl]benzenesulfonamide, 4-(3-cyclopentyloxy-4-methoxy phenyl)-2-oxo-pyrrolidine-1-carboxylic acid 4-isopropenyl cyclohex-1-enylmethyl ester, and (3-methyl 4-oxo-3,4-dihydroimidazo[5,1-d][1,2,3,5]tetrazine-8-carbonyl)carbamic acid-4-isopropenyl cyclohex-1-enylmethyl ester. The details of the chemical reactions generating these compounds are described in the Examples below.

Monoterpenes (or sesquiterpenes) or perillyl alcohol carbamate thereof may be used for the treatment of nervous system cancers, such as a malignant glioma (e.g., astrocytoma, anaplastic astrocytoma, glioblastoma multiforme), retinoblastoma, pilocytic astrocytomas (grade I), meningiomas, metastatic brain tumors, neuroblastoma, pituitary adenomas, skull base meningiomas, and skull base cancer. As used herein, the term “nervous system tumors” refers to a condition in which a subject has a malignant proliferation of nervous system cells.

Cancers that can be treated by the present monoterpene (or sesquiterpene) or perillyl alcohol carbamate compositions include, but are not limited to, lung cancer, ear, nose and throat cancer, leukemia, colon cancer, melanoma, pancreatic cancer, mammary cancer, prostate cancer, breast cancer, hematopoietic cancer, ovarian cancer, basal cell carcinoma, biliary tract cancer; bladder cancer; bone cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia including acute myeloid leukemia, acute lymphoid leukemia, chronic myeloid leukemia, chronic lymphoid leukemia; liver cancer; lymphoma including Hodgkin's and Non-Hodgkin's lymphoma; myeloma; fibroma, neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas. U.S. Pat. No. 7,601,355.

The present invention also provides methods of treating CNS disorders, including, without limitation, primary degenerative neurological disorders such as Alzheimer's, Parkinson's, psychological disorders, psychosis and depression. Treatment may consist of the use of purified monoterpenes or sesquiterpenes alone or in combination with current medications used in the treatment of Parkinson's, Alzheimer's, or psychological disorders. For example, purified monoterpenes or sesquiterpenes may be used as a solvent for the inhalation of current medications used in the treatment of Parkinson's, Alzheimer's, or psychological disorders.

The monoterpene, sesquiterpene or perillyl alcohol carbamate may be used in combination with radiation therapy. In one embodiment, the present invention provides for a method of treating tumor cells, such as malignant glioma cells, with radiation, where the cells are treated with an effective amount of a monoterpene, such as perillyl alcohol, and then exposed to radiation. Monoterpene treatment may be before, during and/or after radiation. For example, the monoterpene or sesquiterpene may be administered continuously beginning one week prior to the initiation of radiotherapy and continued for two weeks after the completion of radiotherapy. U.S. Pat. Nos. 5,587,402 and 5,602,184.

The present monoterpene, sesquiterpene or perillyl alcohol carbamate may be used in combination with at least one therapeutic agents, including, but not limited to, chemotherapeutic agents, immunotherapeutic agents, and antibodies (e.g., monoclonal antibodies). The anti-cancer agents that may be used in combination with the purified monoterpene or sesquiterpene can have one or more of the following effects on cancer cells or the subject: cell death; decreased cell proliferation; decreased numbers of cells; inhibition of cell growth; apoptosis; necrosis; mitotic catastrophe; cell cycle arrest; decreased cell size; decreased cell division; decreased cell survival; decreased cell metabolism; markers of cell damage or cytotoxicity; indirect indicators of cell damage or cytotoxicity such as tumor shrinkage; improved survival of a subject; or disappearance of markers associated with undesirable, unwanted, or aberrant cell proliferation. U.S. Patent Publication No. 20080275057.

Also encompassed by the present invention are admixtures and/or coformulations of a monoterpene (or sesquiterpene) or a perillyl alcohol carbamate and at least one therapeutic agent, including, but not limited to, a chemotherapeutic agent.

Chemotherapeutic agents include, but are not limited to, DNA alkylating agents, topoisomerase inhibitors, endoplasmic reticulum stress inducing agents, a platinum compound, an antimetabolite, vincalkaloids, taxanes, epothilones, enzyme inhibitors, receptor antagonists, therapeutic antibodies, tyrosine kinase inhibitors, boron radiosensitizers (i.e. velcade), and chemotherapeutic combination therapies.

In one embodiment, the present invention provides for a method of treating tumor cells, such as malignant glioma cells, with chemotherapy, where the cells are treated with an effective amount of a monoterpene, such as perillyl alcohol, and then exposed to chemotherapy. Monoterpene treatment may be before, during and/or after chemotherapy.

DNA alkylating agents are well known in the art and are used to treat a variety of tumors. Non-limiting examples of DNA alkylating agents are nitrogen mustards, such as Mechlorethamine, Cyclophosphamide (Ifosfamide, Trofosfamide), Chlorambucil (Melphalan, Prednimustine), Bendamustine, Uramustine and Estramustine; nitrosoureas, such as Carmustine (BCNU), Lomustine (Semustine), Fotemustine, Nimustine, Ranimustine and Streptozocin; alkyl sulfonates, such as Busulfan (Mannosulfan, Treosulfan); Aziridines, such as Carboquone, ThioTEPA, Triaziquone, Triethylenemelamine; Hydrazines (Procarbazine); Triazenes such as Dacarbazine and Temozolomide; Altretamine and Mitobronitol.

Non-limiting examples of Topoisomerase I inhibitors include Campothecin derivatives including CPT-11 (irinotecan), SN-38, APC, NPC, campothecin, topotecan, exatecan mesylate, 9-nitrocamptothecin, 9-aminocamptothecin, lurtotecan, rubitecan, silatecan, gimatecan, diflomotecan, extatecan, BN-80927, DX-8951f, and MAG-CPT as described in Pommier Y. (2006) Nat. Rev. Cancer 6(10):789-802 and U.S. Patent Publication No. 200510250854; Protoberberine alkaloids and derivatives thereof including berberrubine and coralyne as described in Li et al. (2000) Biochemistry 39(24):7107-7116 and Gatto et al. (1996) Cancer Res. 15(12):2795-2800; Phenanthroline derivatives including Benzo[i]phenanthridine, Nitidine, and fagaronine as described in Makhey et al. (2003) Bioorg. Med. Chem. 11 (8): 1809-1820; Terbenzimidazole and derivatives thereof as described in Xu (1998) Biochemistry 37(10):3558-3566; and Anthracycline derivatives including Doxorubicin, Daunorubicin, and Mitoxantrone as described in Foglesong et al. (1992) Cancer Chemother. Pharmacol. 30(2):123-125, Crow et al. (1994) J. Med. Chem. 37(19):31913194, and Crespi et al. (1986) Biochem. Biophys. Res. Commun. 136(2):521-8. Topoisomerase II inhibitors include, but are not limited to Etoposide and Teniposide. Dual topoisomerase I and II inhibitors include, but are not limited to, Saintopin and other Naphthecenediones, DACA and other Acridine-4-Carboxamindes, Intoplicine and other Benzopyridoindoles, TAS-I03 and other 7H-indeno[2,1-c]Quinoline-7-ones, Pyrazoloacridine, XR 11576 and other Benzophenazines, XR 5944 and other Dimeric compounds, 7-oxo-7H-dibenz[f,ij]Isoquinolines and 7-oxo-7H-benzo[e]Perimidines, and Anthracenyl-amino Acid Conjugates as described in Denny and Baguley (2003) Curr. Top. Med. Chem. 3(3):339-353. Some agents inhibit Topoisomerase II and have DNA intercalation activity such as, but not limited to, Anthracyclines (Aclarubicin, Daunorubicin, Doxorubicin, Epirubicin, Idarubicin, Amrubicin, Pirarubicin, Valrubicin, Zorubicin) and Antracenediones (Mitoxantrone and Pixantrone).

Examples of endoplasmic reticulum stress inducing agents include, but are not limited to, dimethyl-celecoxib (DMC), nelfinavir, celecoxib, and boron radiosensitizers (i.e. velcade (Bortezomib)).

Platinum based compound which is a subclass of DNA alkylating agents. Non-limiting examples of such agents include Carboplatin, Cisplatin, Nedaplatin, Oxaliplatin, Triplatin tetranitrate, Satraplatin, Aroplatin, Lobaplatin, and JM-216. (see McKeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

“FOLFOX” is an abbreviation for a type of combination therapy that is used to treat colorectal cancer. It includes 5-FU, oxaliplatin and leucovorin. Information regarding this treatment is available on the National Cancer Institute's web site, cancer.gov, last accessed on Jan. 16, 2008.

“FOLFOX/BV” is an abbreviation for a type of combination therapy that is used to treat colorectal cancer. This therapy includes 5-FU, oxaliplatin, leucovorin and Bevacizumab. Furthermore, “XELOX/BV” is another combination therapy used to treat colorectal cancer, which includes the prodrug to 5-FU, known as Capecitabine (Xeloda) in combination with oxaliplatin and bevacizumab. Information regarding these treatments are available on the National Cancer Institute's web site, cancer.gov or from 23 the National Comprehensive Cancer Network's web site, nccn.org, last accessed on May 27, 2008.

Non-limiting examples of antimetabolite agents include Folic acid based, i.e. dihydrofolate reductase inhibitors, such as Aminopterin, Methotrexate and Pemetrexed; thymidylate synthase inhibitors, such as Raltitrexed, Pemetrexed; Purine based, i.e. an adenosine deaminase inhibitor, such as Pentostatin, a thiopurine, such as Thioguanine and Mercaptopurine, a halogenated/ribonucleotide reductase inhibitor, such as Cladribine, Clofarabine, Fludarabine, or a guanine/guanosine: thiopurine, such as Thioguanine; or Pyrimidine based, i.e. cytosine/cytidine: hypomethylating agent, such as Azacitidine and Decitabine, a DNA polymerase inhibitor, such as Cytarabine, a ribonucleotide reductase inhibitor, such as Gemcitabine, or a thymine/thymidine: thymidylate synthase inhibitor, such as a Fluorouracil (5-FU). Equivalents to 5-FU include prodrugs, analogs and derivative thereof such as 5′-deoxy-5-fluorouridine (doxifluroidine), 1-tetrahydrofuranyl-5-fluorouracil (ftorafur), Capecitabine (Xeloda), S-I (MBMS-247616, consisting of tegafur and two modulators, a 5-chloro-2,4dihydroxypyridine and potassium oxonate), ralititrexed (tomudex), nolatrexed (Thymitaq, AG337), LY231514 and ZD9331, as described for example in Papamicheal (1999) The Oncologist 4:478-487.

Examples of vincalkaloids, include, but are not limited to Vinblastine, Vincristine, Vinflunine, Vindesine and Vinorelbine.

Examples of taxanes include, but are not limited to docetaxel, Larotaxel, Ortataxel, Paclitaxel and Tesetaxel. An example of an epothilone is iabepilone.

Examples of enzyme inhibitors include, but are not limited to farnesyltransferase inhibitors (Tipifamib); CDK inhibitor (Alvocidib, Seliciclib); proteasome inhibitor (Bortezomib); phosphodiesterase inhibitor (Anagrelide; rolipram); IMP dehydrogenase inhibitor (Tiazofurine); and lipoxygenase inhibitor (Masoprocol). Examples of receptor antagonists include, but are not limited to ERA (Atrasentan); retinoid X receptor (Bexarotene); and a sex steroid (Testolactone).

Examples of therapeutic antibodies include, but are not limited to anti-HER1/EGFR (Cetuximab, Panitumumab); Anti-HER2/neu (erbB2) receptor (Trastuzumab); Anti-EpCAM (Catumaxomab, Edrecolomab) Anti-VEGF-A (Bevacizumab); Anti-CD20 (Rituximab, Tositumomab, Ibritumomab); Anti-CD52 (Alemtuzumab); and Anti-CD33 (Gemtuzumab). U.S. Pat. Nos. 5,776,427 and 7,601,355.

Examples of tyrosine kinase inhibitors include, but are not limited to inhibitors to ErbB: HER1/EGFR (Erlotinib, Gefitinib, Lapatinib, Vandetanib, Sunitinib, Neratinib); HER2/neu (Lapatinib, Neratinib); RTK class III: C-kit (Axitinib, Sunitinib, Sorafenib), FLT3 (Lestaurtinib), PDGFR (Axitinib, Sunitinib, Sorafenib); and VEGFR (Vandetanib, Semaxanib, Cediranib, Axitinib, Sorafenib); bcr-abl (Imatinib, Nilotinib, Dasatinib); Src (Bosutinib) and Janus kinase 2 (Lestaurtinib).

Cetuximab is an example of an anti-EGFR antibody. It is a chimeric human/mouse monoclonal antibody that targets the epidermal growth factor receptor (EGFR). Biological equivalent antibodies are identified herein as modified antibodies and those which bind to the same epitope of the EGFR antigen and produce a substantially equivalent biological response such as, preventing ligand binding of the EGFR, preventing activation of the EGFR receptor and the blocking of the downstream signaling of the EGFR pathway resulting in disrupted cell growth.

“Lapatinib” (Tykerb®) is an dual EGFR and erbB-2 inhibitor. Lapatinib has been investigated as an anticancer monotherapy, as well as in combination with trastuzumab, capecitabine, letrozole, paclitaxel and FOLF1R1 (irinotecan, 5-fluorouracil and leucovorin), in a number of clinical trials. It is currently in phase III testing for the oral treatment of metastatic breast, head and neck, lung, gastric, renal and bladder cancer.

A chemical equivalent of lapatinib is a small molecule or compound that is a tyrosine kinase inhibitor (TKI) or alternatively a HER-1 inhibitor or a HER-2 inhibitor. Several TKIs have been found to have effective antitumor activity and have been approved or are in clinical trials. Examples of such include, but are not limited to Zactima (ZD6474), Iressa (gefitinib) and Tarceva (erlotinib), imatinib mesylate (STI571; Gleevec), erlotinib (OSI-1774; Tarceva), canertinib (CI 1033), semaxinib (SU5416), vatalanib (PTK787/ZK222584), sorafenib (BAY 43-9006), sutent (SUI 1248) and lefltmomide (SU101). A biological equivalent of lapatinib is a peptide, antibody or antibody derivative thereof that is a HER-1 inhibitor and/or a HER-2 inhibitor. Examples of such include but are not limited to the humanized antibody trastuzumab and Herceptin.

PTK/ZK is a “small” molecule tyrosine kinase inhibitor with broad specificity that targets all VEGF receptors (VEGFR), the platelet-derived growth factor (PDGF) receptor, c-KIT and c-Fms. Drevs (2003) Idrugs 6(8):787-794. PTK/ZK is a targeted drug that blocks angiogenesis and lymphangiogenesis by inhibiting the activity of all known receptors that bind VEGF including VEGFR-I (Flt-1), VEGFR-2 (KDR/Flk-1) and VEGFR-3 (Flt-4). The chemical names of PTK/ZK are 1-[4-Chloroanilino]-4-[4-pyridylmethyl] phthalazine Succinate or 1-Phthalazinamine, N-(4-chlorophenyl)-4-(4-pyridinylmethyl)-butanedioate (1:1). Synonyms and analogs of PTK/TK are known as Vatalanib, CGP79787D, PTK787/ZK 222584, CGP-79787, DE-00268, PTK-787, PTK787A, VEGFR-TK inhibitor, ZK 222584 and ZK.

Chemotherapeutic agents that can be used in combination with the purified monoterpenes, sesquiterpenes or perillyl alcohol carbamate may also include amsacrine, Trabectedin, retinoids (Alitretinoin, Tretinoin), Arsenic trioxide, asparagine depleter Asparaginase/Pegaspargase), Celecoxib, Demecolcine, Elesclomol, Elsamitrucin, Etoglucid, Lonidamine, Lucanthone, Mitoguazone, Mitotane, Oblimersen, Temsirolimus, and Vorinostat.

The compositions and methods of the present invention may be used to decrease the level of the Ras protein. The Ras family is a protein family of small GTPases that are involved in cellular signal transduction. Activation of Ras signaling causes cell growth, differentiation and survival. Mtations in ras genes can permanently activate it and cause inappropriate transmission inside the cell even in the absence of extracellular signals. Because these signals result in cell growth and division, dysregulated Ras signaling can ultimately lead to oncogenesis and cancer. Activating mutations in Ras are found in 20-25% of all human tumors and up to 90% in specific tumor types. Goodsell D S (1999). Downward J., “The molecular perspective: the ras oncogene”. Oncologist 4 (3): 263-4. (January 2003). “Targeting RAS signalling pathways in cancer therapy”. Nat. Rev. Cancer 3 (1): 11-22. Ras family members include, but are not limited to, HRAS; KRAS; NRAS; DIRAS1; DIRAS2; DIRAS3; ERAS; GEM; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; and RRAS. Wennerberg K, Rossman K L, Der C J (March 2005). “The Ras superfamily at a glance”. J. Cell. Sci. 118 (Pt 5): 843-6.

The compositions and methods of the present invention may be used to increase paracellular permeability, for example, paracellular permeability of endothelial cells or epithelial cells. The present compositions and methods may be used to increase blood brain barrier permeability.

The compositions and methods of the present invention may be used to decrease or inhibit angiogenesis. The present compositions and methods may decrease or inhibit production of pro-angiogenic cytokines, including, but not limited to, vascular endothelial growth factor (VEGF) and interleukin 8 (IL8).

The purified monoterpenes, sesquiterpenes or perillyl alcohol carbamate may be used in combination with angiogenesis inhibitors. Examples of angiogenesis inhibitors include, but are not limited to, angiostatin, angiozyme, antithrombin III, AG3340, VEGF inhibitors (e.g., anti-VEGF antibody), batimastat, bevacizumab (avastin), BMS-275291, CAI, 2C3, HuMV833 Canstatin, Captopril, carboxyamidotriazole, cartilage derived inhibitor (CDI), CC-5013, 6-O-(chloroacetyl-carbonyl)-fumagillol, COL-3, combretastatin, combretastatin A4 Phosphate, Dalteparin, EMD 121974 (Cilengitide), endostatin, erlotinib, gefitinib (Iressa), genistein, halofuginone hydrobromide, Id1, Id3, IM862, imatinib mesylate, IMC-IC11 Inducible protein 10, interferon-alpha, interleukin 12, lavendustin A, LY317615 or AE-941, marimastat, mspin, medroxpregesterone acetate, Meth-1, Meth-2, 2-methoxyestradiol (2-ME), neovastat, oteopontin cleaved product, PEX, pigment epithelium growth factor (PEGF), platelet factor 4, prolactin fragment, proliferin-related protein (PRP), PTK787/ZK 222584, ZD6474, recombinant human platelet factor 4 (rPF4), restin, squalamine, SU5416, SU6668, SU11248 suramin, Taxol, Tecogalan, thalidomide, thrombospondin, TNP-470, troponin-1, vasostatin, VEG1, VEGF-Trap, and ZD6474.

Non-limiting examples of angiogenesis inhibitors also include, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, pentosan polysulfate, angiotensin II antagonists, cyclooxygenase inhibitors (including non-steroidal anti-inflammatory drugs (NSAIDs) such as aspirin and ibuprofen, as well as selective cyclooxygenase-2 inhibitors such as celecoxib and rofecoxib), and steroidal anti-inflammatories (such as corticosteroids, mineralocorticoids, dexamethasone, prednisone, prednisolone, methylpred, betamethasone).

Other therapeutic agents that modulate or inhibit angiogenesis and may also be used in combination with the compounds of the instant invention include agents that modulate or inhibit the coagulation and fibrinolysis systems. Examples of such agents that modulate or inhibit the coagulation and fibrinolysis pathways include, but are not limited to, heparin, low molecular weight heparins and carboxypeptidase U inhibitors (also known as inhibitors of active thrombin activatable fibrinolysis inhibitor [TAFIa]). U.S. Patent Publication No. 20090328239. U.S. Pat. No. 7,638,549.

The present invention also provides a method of improving immunomodulatory therapy responses comprising the steps of exposing cells to an effective amount of a monoterpene or sisquiterpene, such as perillyl alcohol, before or during immunomodulatory treatment. Preferred immunomodulatory agents are cytokines, such interleukins, lymphokines, monokines, interfereons and chemokines.

This invention further provides for compositions where the purified monoterpene (or sesquiterpene) functions as a solvent or a permeation enhancer. In one aspect, the monoterpene is perillyl alcohol. Examples of the therapeutic agents are provided infra. The composition may further comprise one or more pharmaceutically acceptable carriers, co-solvents, or other permeation enhancers.

In one embodiment, the composition contains the following components: a therapeutic agent; at least about 0.03% (v/v) of a monoterpene (or sesquiterpene) such as perillyl alcohol; at least about 2.6% (v/v) of a co-solvent which can be 1.3% (v/v) of a polyol such as glycerol or an equivalent thereof; and at least about 1.3% (v/v) of ethanol or an equivalent thereof.

Other permeation enhancers that may be used together with the purified monoterpene (or sesquiterpene) include, but are not limited to, fatty acid esters of glycerin, such as capric, caprylic, dodecyl, oleic acids; fatty acid esters of isosorbide, sucrose, polyethylene glycol; caproyllactylic acid; laureth-2; laureth-2 acetate; laureth-2 benzoate; laureth-3 carboxylic acid; laureth-4; laureth-5 carboxylic acid; oleth-2; glyceryl pyroglutamate oleate; glyceryl oleate; N-lauroyl sarcosine; N-myristoyl sarcosine; Noctyl-2-pyrrolidone; lauraminopropionic acid; polypropylene glycol-4-laureth-2; polypropylene glycol-4-laureth-5dimethyl lauramide; lauramide diethanolamine (DEA), lauryl pyroglutamate (LP), glyceryl monolaurate (GML), glyceryl monocaprylate, glyceryl monocaprate, glyceryl monooleate (GMO) and sorbitan monolaurate. Polyols or ethanol may act as a permeation enhancer or co-solvent. See U.S. Pat. Nos. 5,785,991; 5,843,468; 5,882,676; and 6,004,578 for additional permeation enhancers.

Co-solvents are well-known in the art and include, without limitation, glycerol, polyethylene glycol (PEG), glycol, ethanol, methanol, propanol, isopropanol, butanol and the like.

The present composition may be administered by any method known in the art, including, without limitation, intranasal, oral, ocular, intraperitoneal, inhalation, intravenous, ICV, intracisternal injection or infusion, subcutaneous, implant, vaginal, sublingual, urethral (e.g., urethral suppository), subcutaneous, intramuscular, intravenous, transdermal, rectal, sub-lingual, mucosal, ophthalmic, spinal, intrathecal, intra-articular, intra-arterial, sub-arachinoid, bronchial and lymphatic administration. Topical formulation may be in the form of gel, ointment, cream, aerosol, etc; intranasal formulation can be delivered as a spray or in a drop; transdermal formulation may be administered via a transdermal patch or iontorphoresis; inhalation formulation can be delivered using a nebulizer or similar device. Compositions can also take the form of tablets, pills, capsules, semisolids, powders, sustained release formulations, solutions, suspensions, elixirs, aerosols, or any other appropriate compositions.

To prepare such pharmaceutical compositions, one or more of the purified monoterpenes (or sesquiterpenes) or perillyl alcohol carbamate may be mixed with a pharmaceutical acceptable carrier, adjuvant and/or excipient, according to conventional pharmaceutical compounding techniques. Pharmaceutically acceptable carriers that can be used in the present compositions encompass any of the standard pharmaceutical carriers, such as a phosphate buffered saline solution, water, and emulsions, such as an oil/water or water/oil emulsion, and various types of wetting agents. The compositions can additionally contain solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols. For examples of carriers, stabilizers and adjuvants, see Remington's Pharmaceutical Sciences, edited by E. W. Martin (Mack Publishing Company, 18th ed., 1990). The compositions also can include stabilizers and preservatives.

As used herein, the term “therapeutically effective amount” is an amount sufficient to treat a specified disorder or disease or alternatively to obtain a pharmacological response treating a disorder or disease. Methods of determining the most effective means and dosage of administration can vary with the composition used for therapy, the purpose of the therapy, the target cell being treated, and the subject being treated. Treatment dosages generally may be titrated to optimize safety and efficacy. Single or multiple administrations can be carried out with the dose level and pattern being selected by the treating physician. Suitable dosage formulations and methods of administering the agents can be readily determined by those of skill in the art. For example, the compositions are administered at about 0.01 mg/kg to about 200 mg/kg, about 0.1 mg/kg to about 100 mg/kg, or about 0.5 mg/kg to about 50 mg/kg. When the compounds described herein are co-administered with another agent or therapy, the effective amount may be less than when the agent is used alone.

This invention also provides the compositions as described above for intranasal administration. As such, the compositions can further comprise a permeation enhancer. Southall et al. Developments in Nasal Drug Delivery, 2000. The purified monoterpene (or sesquiterpene) or perillyl alcohol carbamate derivative may be administered intranasally in a liquid form such as a solution, an emulsion, a suspension, drops, or in a solid form such as a powder, gel, or ointment. Devices to deliver intranasal medications are well known in the art. Nasal drug delivery can be carried out using devices including, but not limited to, intranasal inhalers, intranasal spray devices, atomizers, nasal spray bottles, unit dose containers, pumps, droppers, squeeze bottles, nebulizers, metered dose inhalers (MDI), pressurized dose inhalers, insufflators, and bi-directional devices. The nasal delivery device can be metered to administer an accurate effective dosage amount to the nasal cavity. The nasal delivery device can be for single unit delivery or multiple unit delivery. In a specific example, the ViaNase Electronic Atomizer from Kurve Technology (Bethell, Wash.) can be used in this invention (http://www.kurvetech.com). The compounds of the present invention may also be delivered through a tube, a catheter, a syringe, a packtail, a pledget, a nasal tampon or by submucosal infusion. U.S. Patent Publication Nos. 20090326275, 20090291894, 20090281522 and 20090317377.

The purified monoterpene (or sesquiterpene) or perillyl alcohol carbamate derivative can be formulated as aerosols using standard procedures. The monoterpene (or sesquiterpene) may be formulated with or without solvents, and formulated with or without carriers. The formulation may be a solution, or may be an aqueous emulsion with one or more surfactants. For example, an aerosol spray may be generated from pressurized container with a suitable propellant such as, dichlorodifluoromethane, trichlorofluoromethane, dichlorotetrafluoroethane, hydrocarbons, compressed air, nitrogen, carbon dioxide, or other suitable gas. The dosage unit can be determined by providing a valve to deliver a metered amount. Pump spray dispensers can dispense a metered dose or a dose having a specific particle or droplet size. As used herein, the term “aerosol” refers to a suspension of fine solid particles or liquid solution droplets in a gas. Specifically, aerosol includes a gas-borne suspension of droplets of a monoterpene (or sesquiterpene), as may be produced in any suitable device, such as an MDI, a nebulizer, or a mist sprayer. Aerosol also includes a dry powder composition of the composition of the instant invention suspended in air or other carrier gas. Gonda (1990) Critical Reviews in Therapeutic Drug Carrier Systems 6:273-313. Raeburn et al., (1992) Pharmacol. Toxicol. Methods 27:143-159.

The purified monoterpene (or sesquiterpene) or perillyl alcohol carbamate may be delivered to the nasal cavity as a powder in a form such as microspheres delivered by a nasal insufflator. The monoterpene (or sesquiterpene) may be absorbed to a solid surface, for example, a carrier. The powder or microspheres may be administered in a dry, air-dispensable form. The powder or microspheres may be stored in a container of the insufflator. Alternatively, the powder or microspheres may be filled into a capsule, such as a gelatin capsule, or other single dose unit adapted for nasal administration.

The pharmaceutical composition can be delivered to the nasal cavity by direct placement of the composition in the nasal cavity, for example, in the form of a gel, an ointment, a nasal emulsion, a lotion, a cream, a nasal tampon, a dropper, or a bioadhesive strip. In certain embodiments, it can be desirable to prolong the residence time of the pharmaceutical composition in the nasal cavity, for example, to enhance absorption. Thus, the pharmaceutical composition can optionally be formulated with a bioadhesive polymer, a gum (e.g., xanthan gum), chitosan (e.g., highly purified cationic polysaccharide), pectin (or any carbohydrate that thickens like a gel or emulsifies when applied to nasal mucosa), a microsphere (e.g., starch, albumin, dextran, cyclodextrin), gelatin, a liposome, carbamer, polyvinyl alcohol, alginate, acacia, chitosans and/or cellulose (e.g., methyl or propyl; hydroxyl or carboxy; carboxymethyl or hydroxylpropyl).

The composition containing the purified monoterpene (or sesquiterpene) or perillyl alcohol carbamate can be administered by oral inhalation into the respiratory tract, i.e., the lungs.

Typical delivery systems for inhalable agents include nebulizer inhalers, dry powder inhalers (DPI), and metered-dose inhalers (MDI).

Nebulizer devices produce a stream of high velocity air that causes a therapeutic agent in the form of liquid to spray as a mist. The therapeutic agent is formulated in a liquid form such as a solution or a suspension of particles of suitable size. In one embodiment, the particles are micronized. The term “micronized” is defined as having about 90% or more of the particles with a diameter of less than about 10.mu.m. Suitable nebulizer devices are provided commercially, for example, by PARI GmbH (Starnberg, Germany). Other nebulizer devices include Respimat (Boehringer Ingelheim) and those disclosed in, for example, U.S. Pat. Nos. 7,568,480 and 6,123,068, and WO 97/12687. The monoterpenes (or sesquiterpenes) can be formulated for use in a nebulizer device as an aqueous solution or as a liquid suspension.

DPI devices typically administer a therapeutic agent in the form of a free-flowing powder that can be dispersed in a patient's air-stream during inspiration. DPI devices which use an external energy source may also be used in the present invention. In order to achieve a free-flowing powder, the therapeutic agent can be formulated with a suitable excipient (e.g., lactose). A dry powder formulation can be made, for example, by combining dry lactose having a particle size between about 1.mu.m and 100.mu.m with micronized particles of the monoterpenes (or sesquiterpenes) and dry blending. Alternatively, the monoterpene can be formulated without excipients. The formulation is loaded into a dry powder dispenser, or into inhalation cartridges or capsules for use with a dry powder delivery device. Examples of DPI devices provided commercially include Diskhaler (GlaxoSmithKline, Research Triangle Park, N.C.) (see, e.g., U.S. Pat. No. 5,035,237); Diskus (GlaxoSmithKline) (see, e.g., U.S. Pat. No. 6,378,519; Turbuhaler (AstraZeneca, Wilmington, Del.) (see, e.g., U.S. Pat. No. 4,524,769); and Rotahaler (GlaxoSmithKline) (see, e.g., U.S. Pat. No. 4,353,365). Further examples of suitable DPI devices are described in U.S. Pat. Nos. 5,415,162, 5,239,993, and 5,715,810 and references therein.

MDI devices typically discharge a measured amount of therapeutic agent using compressed propellant gas. Formulations for MDI administration include a solution or suspension of active ingredient in a liquefied propellant. Examples of propellants include hydrofluoroalklanes (HFA), such as 1,1,1,2-tetrafluoroethane (HFA 134a) and 1,1,1,2,3,3,3-heptafluoro-n-propane, (HFA 227), and chlorofluorocarbons, such as CCl.sub.3F. Additional components of HFA formulations for MDI administration include co-solvents, such as ethanol, pentane, water; and surfactants, such as sorbitan trioleate, oleic acid, lecithin, and glycerin. (See, for example, U.S. Pat. No. 5,225,183, EP 0717987, and WO 92/22286). The formulation is loaded into an aerosol canister, which forms a portion of an MDI device. Examples of MDI devices developed specifically for use with HFA propellants are provided in U.S. Pat. Nos. 6,006,745 and 6,143,227. For examples of processes of preparing suitable formulations and devices suitable for inhalation dosing see U.S. Pat. Nos. 6,268,533, 5,983,956, 5,874,063, and 6,221,398, and WO 99/53901, WO 00/61108, WO 99/55319 and WO 00/30614.

The monoterpenes (or sesquiterpenes) or perillyl alcohol carbamate may be encapsulated in liposomes or microcapsules for delivery via inhalation. A liposome is a vesicle composed of a lipid bilayer membrane and an aqueous interior. The lipid membrane may be made of phospholipids, examples of which include phosphatidylcholine such as lecithin and lysolecithin; acidic phospholipids such as phosphatidylserine and phosphatidylglycerol; and sphingophospholipids such as phosphatidylethanolamine and sphingomyelin. Alternatively, cholesterol may be added. A microcapsule is a particle coated with a coating material. For example, the coating material may consist of a mixture of a film-forming polymer, a hydrophobic plasticizer, a surface activating agent or/and a lubricant nitrogen-containing polymer. U.S. Pat. Nos. 6,313,176 and 7,563,768.

Because of their ability to easily penetrate the dermis, monoterpenes may also be used alone or in combination with other chemotherapeutic agents via topical application for the treatment of localized cancers such as breast cancer or melanomas. As a transdermal delivery agent, monoterpenes may also be used in combination with narcotics or analgesics for transdermal delivery of pain medication.

This invention also provides the compositions as described above for ocular administration. As such, the compositions can further comprise a permeation enhancer. For ocular administration, the compositions described herein can be formulated as a solution, emulsion, suspension, etc. A variety of vehicles suitable for administering compounds to the eye are known in the art. Specific non-limiting examples are described in U.S. Pat. Nos. 6,261,547; 6,197,934; 6,056,950; 5,800,807; 5,776,445; 5,698,219; 5,521,222; 5,403,841; 5,077,033; 4,882,150; and 4,738,851.

The monoterpenes (or sesquiterpenes) or perillyl alcohol carbamate can be given alone or in combination with other drugs for the treatment of the above diseases for a short or prolonged period of time. The present compositions can be administered to a mammal, preferably a human. Mammals include, but are not limited to, murines, rats, rabbit, simians, bovines, ovine, porcine, canines, feline, farm animals, sport animals, pets, equine, and primates.

The present invention further provides an article of manufacture (such as a kit) comprising the purified monoterpene (or sesquiterpene) formulated for intranasal administration, and a device for intranasal administration of the purified monoterpene (or sesquiterpene). The device for intranasal administration may be an intranasal spray device, an atomizer, a nebulizer, a metered dose inhaler (MDI), a pressurized dose inhaler, an insufflator, an intranasal inhaler, a nasal spray bottle, a unit dose container, a pump, a dropper, a squeeze bottle, or a bi-directional device. The article of manufacture can contain printed matter indicating purified monoterpene (or sesquiterpene) is to be used to treat a disease, such as cancer or other nervous system disorders. The printed matter may state that the monoterpenes (or sesquiterpenes) may be administered alone, or in combination with radiation, surgery or chemotherapeutic agents. The monoterpene or sesquiterpene may also be co-administered with antiviral agents, anti-inflammatory agents or antibiotics. The agents may be administered concurrently or sequentially.

The invention also provides a method for inhibiting the growth of a cell in vitro, ex vivo or in vivo, where a cell, such as a cancer cell, is contacted with an effective amount of the purified monoterpene (or sesquiterpene) as described herein. The present compositions and methods may be used to inhibit the growth of a cell that is resistant to a chemotherapeutic agent. For example, the present compositions and methods may be used to inhibit the growth of a temozolomide-resistant cell.

Pathological cells or tissue such as hyperproliferative cells or tissue may be treated by contacting the cells or tissue with an effective amount of a composition of this invention. The cells, such as cancer cells, can be primary cancer cells or can be cultured cells available from tissue banks such as the American Type Culture Collection (ATCC). The pathological cells can be cells of a systemic cancer, gliomas, meningiomas, pituitary adenomas, or a CNS metastasis from a systemic cancer, lung cancer, prostate cancer, breast cancer, hematopoietic cancer or ovarian cancer. The cells can be from a vertebrate, preferably a mammal, more preferably a human. U.S. Patent Publication No. 2004/0087651. Balassiano et al. (2002) Intern. J. Mol. Med. 10:785-788. Thorne, et al. (2004) Neuroscience 127:481-496. Fernandes, et al. (2005) Oncology Reports 13:943-947. Da Fonseca, et al. (2008) Surgical Neurology 70: 259267. Da Fonseca, et al. (2008) Arch. Immunol. Ther. Exp. 56:267-276. Hashizume, et al. (2008) Neuroncology 10:112-120.

Cancer stem cells (CSCs) or tumour initiating cells are immature cells with stem cell features such as self-renewal. However, self-renewal is exacerbated in CSCs. Reya et al., Stem cells, cancer, and cancer stem cells. Nature. 2001, 414(6859):105-11. Additionally, glioma CSCs are resistant to chemo- and radio-therapy. Bao et al., Glioma stem cells promote radioresistance by preferential activation of the DNA damage response. Nature. 2006, 444(7120):756-60. Rich et al., Chemotherapy and cancer stem cells. Cell Stem Cell. 2007; 1(4):353-5. The present compositions and methods may be used to inhibit the growth of a cancer stem cell, including, but not limited to, a glioblastoma cancer stem cell.

In vitro efficacy of the present composition can be determined using methods well known in the art. For example, the cytoxicity of the present monoterpene (or sesquiterpene) and/or the therapeutic agents may be studied by MTT [3-(4,5-dimethylthiazol-2-yl)-2,5-diphenyl tetrazolium bromide] cytotoxicity assay. MTT assay is based on the principle of uptake of MTT, a tetrazolium salt, by metabolically active cells where it is metabolized into a blue colored formazon product, which can be read spectrometrically. J. of Immunological Methods 65: 55 63, 1983. The cytoxicity of the present monoterpene (or sesquiterpene) and/or the therapeutic agents may be studied by colony formation assay. Functional assays for inhibition of VEGF secretion and IL-8 secretion may be performed via ELISA. Cell cycle block by the present monoterpene (or sesquiterpene) and/or the therapeutic agents may be studied by standard propidium iodide (PI) staining and flow cytometry. Invasion inhibition may be studied by Boyden chambers. In this assay a layer of reconstituted basement membrane, Matrigel, is coated onto chemotaxis filters and acts as a barrier to the migration of cells in the Boyden chambers. Only cells with invasive capacity can cross the Matrigel barrier. Other assays include, but are not limited to cell viability assays, apoptosis assays, and morphological assays.

The following examples are presented for the purposes of illustration only and are not limiting the invention.

Example 1 (S)-Perillyl Alcohol Purification Via 3,5-Dinitrobenzoate Ester (S)-Perillyl alcohol can be purified directly from natural products, or be obtained by synthetic modification of natural products such as beta-pinene (extracted from pine trees) by oxidation and rearrangement (Scheme 1).

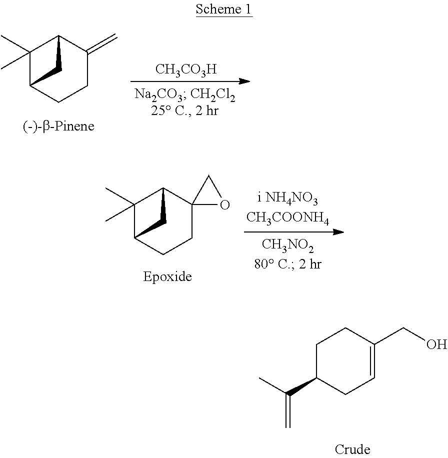

Such sources of (S)-perillyl alcohol are inevitably contaminated by isomers of the target compound which are very similar in physicochemical properties, and therefore, are difficult to remove by conventional methods of purification such as fractional distillation or chromatography.

In this Example, in order to purify (S)-perillyl alcohol from the contaminants that typically accompany it from natural product and/or synthetic sources, perillyl alcohol was first derivatized as its 3,5-dinitrobenzoate ester, which was separated from contaminants by conventional crystallization. Once the derivatized (S)-perillyl alcohol has been purified by crystallization it can then be hydrolyzed to recover the purified (S)-perillyl alcohol (Scheme 2). The purified (S)-perillyl alcohol prepared in this way has a purity greater than about 99%.

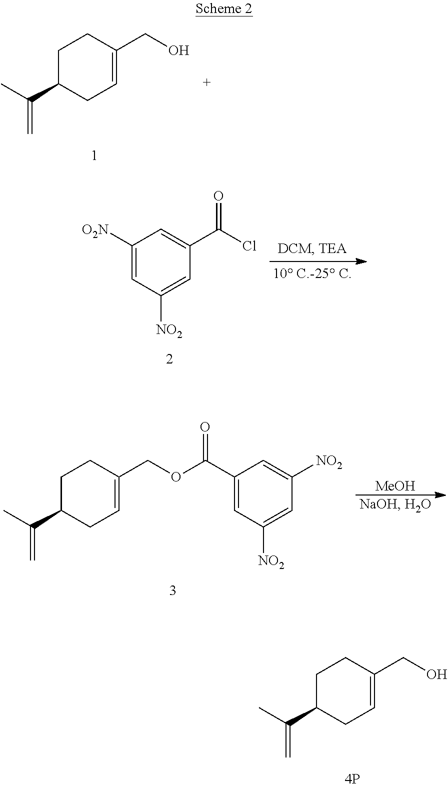

### Synthesis of 3,5-Dinitrobenzoic acid 4-(S)-isopropenyl cyclohex-1-enylmethyl ester (Compound 3)

Triethyl amine (12.2 mL, 87.5 mmol) was added to a mixture of (S)-perillyl alcohol (1, 89.5% 10.0 g, 58.7 mmol) in dichloromethane (70 ml) over a period of 0.25 h while maintaining the temperature below 15° C. The reaction mixture was stirred for 30 min at room temperature. A solution of 3,5-dinitro benzoyl chloride (Compound 2, 14.23 g, 61.7 mmol) dissolved in dichloromethane (30 mL) was added over a period of 0.5 h while keeping the temperature below 15° C. The reaction mixture was allowed to warm to room temperature and then stirred for 3.0 h. The reaction mixture was quenched with water (75 mL) and the organic layer was separated. The aqueous layer was extracted with dichloromethane (50 mL). The combined organic layer was washed with water (2.times.100 mL) and dried over sodium sulphate (25 g). The filtered organic layer was concentrated, and the resulting residue was crystallized from diisopropyl ether (200 mL) to get pure compound 3 (Weight: 14.45 g). The mother liquor was concentrated to half of its volume and 2.1 g was obtained as a second crop. (Total yield: 81.2%, Purity: 99.4% by HPLC.)

### Hydrolysis of 3,5-Dinitrobenzoic acid 4-(S)-isopropenyl cyclohex-1-enylmethyl ester (Compound 3)

Aqueous sodium hydroxide (3.23 g, 80.0 mmol, dissolved in 28 mL of water) was added to an ice cold solution of 3,5-Dinitro-benzoic acid 4-isopropenyl-cyclohex-1-enylmethyl ester (14.0 g, 40.4 mmol) in methanol (140 mL) over a period of 0.25 h. The reaction mixture was allowed to warm to room temperature and then stirred for 3.0 h. The methanol was concentrated under vacuum and the resulting residue was suspended in water (60 mL) and extracted with ethyl acetate (2×100 mL). The organic layer was washed with water (2×100 mL) followed by brine (15%, 100 mL) and dried over sodium sulphate (30 g). The filtered organic layer was concentrated under vacuum to get pure (S)-perillyl alcohol (Weight: 5.84 g, Yield: 95% Purity: 99.4% by GC).

Example 2 Synthesis of 3,5-Dinitrobenzoic Acid 4(S)-isopropenyl cyclohex-1-enylmethyl Ester (Compound 3) and Purification by Preparative Chromatography Triethyl amine (5.3 mL, 38.0 mmol) was added to a mixture of (S)-perillyl alcohol (89.5% 5.0 g, 29.3 mmol) in dichloromethane (40 ml) over a period of 0.25 h while maintaining the temperature below 15° C. The reaction mixture was stirred for 30 min at room temperature. A solution of 3,5-dinitro benzoyl chloride (7.43 g, 32.2 mmol) dissolved in dichloromethane (15 mL) was added over a period of 0.5 h while maintain the temperature between 15-20.° C. The reaction mixture was allowed to warm to room temperature and then stirred for 3.0 h. The reaction mixture was quenched with water (40 mL) and the organic layer was separated. The aqueous layer was extracted with dichloromethane (25 mL). The combined organic layer was washed with water (2.times.50 mL) and dried over sodium sulphate (20 g). The filtered organic layer was concentrated under vacuum to give a residue which was purified by column chromatography. Column dimensions were as follows: diameter: 2.5 cm, height: 30 cm, silica: 200 mesh. The column was eluted with hexanes:ethyl acetate (98:2, 200 mL) followed by hexanes: ethyl acetate (95:5). Based on TLC analysis of the fractions (solvent system; hexanes: ethyl acetate (90:10)), the hexanes: ethyl acetate (95:5) fractions were combined and concentrated under vacuum to give a solid. (Weight: 7.9 g Yield: 78%).

### Example 2 Synthesis of 4-Nitrobenzoic acid 4(S)-isopropenyl cyclohex-1-enylmethyl Ester (Scheme 3)

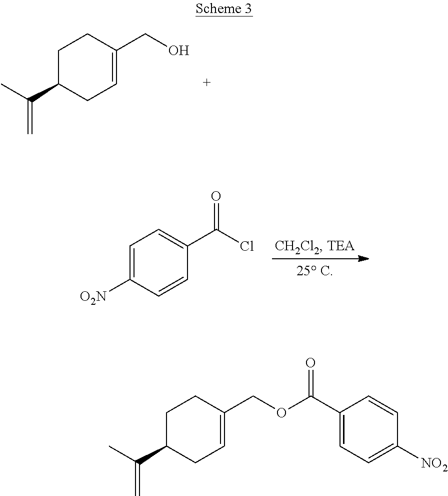 [SMILES: C=C(C)[C@@H]1CC=C(CO)CC1 ; O=C(Cl)c1ccc([N+](=O)[O-])cc1 ; C=C(C)[C@@H]1CC=C(COC(=O)c2ccc([N+](=O)[O-])cc2)CC1]

Triethyl amine (5.92 mL, 42.4 mmol) was added to a mixture of (S)-perillyl alcohol (5.0 g, 32.8 mmol) in dichloromethane (30 ml) over a period of 0.25 h while maintaining the temperature below 15° C. The reaction mixture was stirred for 30 min at room temperature. A solution of 4-nitrobenzoyl chloride (6.39 g, 34.4 mmol) dissolved in dichloromethane (30 mL) was added over a period of 0.5 h while keeping the temperature below 15° C. The reaction mixture was allowed to warm to room temperature and then stirred for 3.0 h. The reaction mixture was quenched with water (50 mL) and the organic layer was separated. The aqueous layer was extracted with dichloromethane (25 mL). The combined organic layer was washed with water (2×0.50 mL) and dried over sodium sulphate (20 g). The filtered organic layer was concentrated to give an oil (Weight: 8.9 g, yield: 90%).

### Example 3 Synthesis of 4-chlorobenzoic Acid 4(S)-isopropenyl cyclohex-1-enylmethyl Ester (Scheme 4)

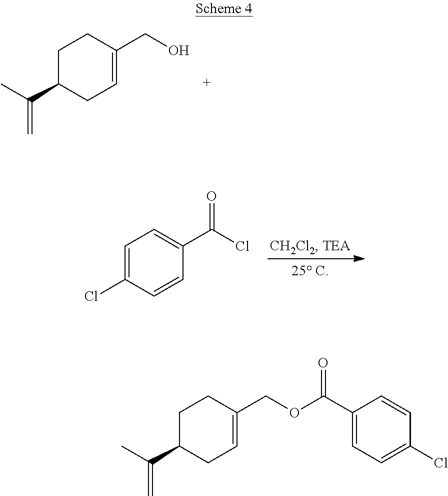 [SMILES: C=C(C)[C@@H]1CC=C(CO)CC1 ; O=C(Cl)c1ccc(Cl)cc1 ; C=C(C)[C@@H]1CC=C(COC(=O)c2ccc(Cl)cc2)CC1]

Triethyl amine (2.85 mL, 20.5 mmol) was added to a mixture of (S)-perillyl alcohol (2.5 g, 16.4 mmol) in dichloromethane (25 ml) over a period of 0.25 h while maintaining the temperature below 15.degree. C. The reaction mixture was stirred for 30 min at room temperature. A solution of 4-chlorobenzoyl chloride (3.01 g, 17.2 mmol) dissolved in dichloromethane (10 mL) was added over a period of 0.5 h while keeping the temperature below 15° C. The reaction mixture was allowed to warm to room temperature and then stirred for 3.0 h. The reaction mixture was quenched with water (30 mL) and the organic layer was separated. The aqueous layer was extracted with dichloromethane (25 mL). The combined organic layer was washed with water (2×30 mL) and dried over sodium sulphate (15 g). The filtered organic layer was concentrated to give an oil (Weight: 3.8 g, yield: 81.7%).

### Example 4 Synthesis of 3,4,5-trimethoxybenzoic Acid 4(S)-isopropenyl cyclohex-1-enylmethyl Ester (Scheme 5)

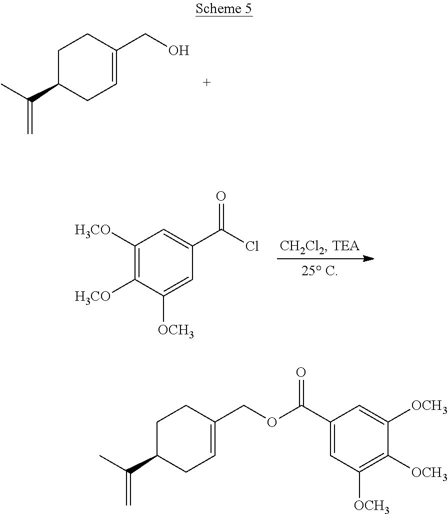 [SMILES: C=C(C)[C@@H]1CC=C(CO)CC1 ; COc1cc(C(=O)Cl)cc(OC)c1OC ; C=C(C)[C@@H]1CC=C(COC(=O)c2cc(OC)c(OC)c(OC)c2)CC1]

Triethyl amine (2.85 mL, 20.5 mmol) was added to a mixture of (S)-perillyl alcohol (2.5 g, 16.4 mmol) in dichloromethane (25 ml) over a period of 0.25 h while maintaining the temperature below 15° C. The reaction mixture was stirred for 30 min at room temperature. A solution of 3,4,5-trimethoxybenzoyl chloride (3.97 g, 17.2 mmol) dissolved in dichloromethane (10 mL) was added over a period of 0.5 h while keeping the temperature below 15° C. The reaction mixture was allowed to warm to room temperature and then stirred for 3.0 h. The reaction mixture was quenched with water (30 mL) and the organic layer was separated. The aqueous layer was extracted with dichloromethane (25 mL). The combined organic layer was washed with water (2×30 mL) and dried over sodium sulphate (15 g). The filtered organic layer was concentrated to give an oil (Weight: 4.8 g, yield: 84.6%).

### Example 5 Synthesis of 4-trimethoxybenzoic Acid 4(S)-isopropenyl cyclohex-1-enylmethyl Ester (Scheme 6)

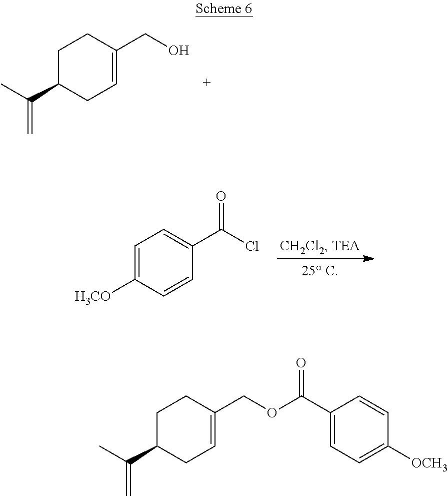 [SMILES: C=C(C)[C@@H]1CC=C(CO)CC1 ; COc1ccc(C(=O)Cl)cc1 ; C=C(C)[C@@H]1CC=C(COC(=O)c2ccc(OC)cc2)CC1]

Triethyl amine (2.97 mL, 21.3 mmol) was added to a mixture of (S)-perillyl alcohol (2.5 g, 16.4 mmol) in dichloromethane (25 ml) over a period of 0.25 h while maintaining the temperature below 15° C. The reaction mixture was stirred for 30 min at room temperature. A solution of 4-methoxybenzoyl chloride (2.94 g, 17.2 mmol) dissolved in dichloromethane (10 mL) was added over a period of 0.5 h while keeping the temperature below 15° C. The reaction mixture was allowed to warm to room temperature and then stirred for 3.0 h. The reaction mixture was quenched with water (30 mL) and the organic layer was separated. The aqueous layer was extracted with dichloromethane (25 mL). The combined organic layer was washed with water (2×30 mL) and dried over sodium sulphate (15 g). The filtered organic layer was concentrated to give an oil (Weight: 4.1 g, yield: 87%).

### Example 6—Synthesis of Dimethyl Celecoxib bisPOH Carbamate (4-(bis-N,N′-4-isopropenyl cyclohex-1-enylmethyloxy carbonyl[5-(2,5-dimethyl phenyl)-3-trifluoromethylpyrazol-1-yl]benzenesulfonamide)

The reaction scheme is the following:

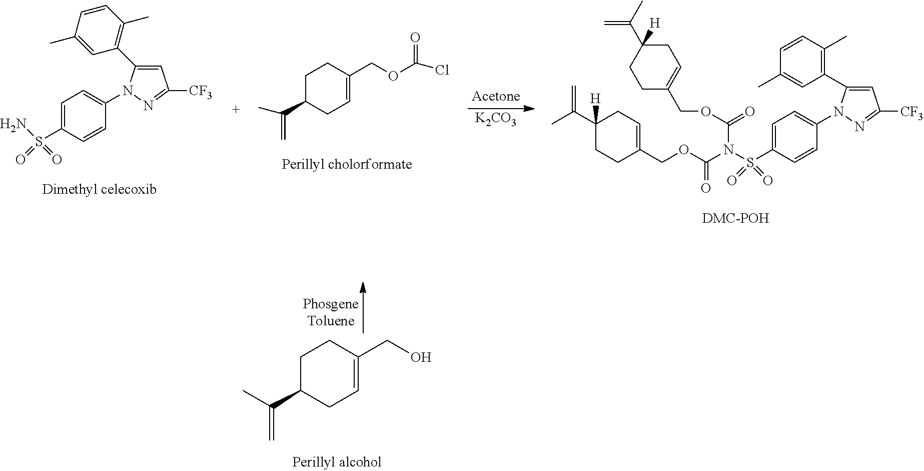 [SMILES: Cc1ccc(C)c(-c2cc(C(F)(F)F)nn2-c2ccc(S(N)(=O)=O)cc2)c1 ; C=C(C)[C@@H]1CC=C(COC(=O)Cl)CC1 ; "K2CO3" ; C=C(C)[C@@H]1CC=C(COC(=O)N(C(=O)OCC2=CC[C@@H](C(=C)C)CC2)S(=O)(=O)c2ccc(-n3nc(C(F)(F)F)cc3-c3cc(C)ccc3C)cc2)CC1 ; C=C(C)[C@@H]1CC=C(CO)CC1]

Phosgene (20% in toluene, 13 ml, 26.2 mmol) was added to a mixture of perillyl alcohol (2.0 grams, 13.1 mmol) and potassium carbonate (5.4 grams, 39.1 mmol) in dry toluene (30 mL) over a period of 30 minutes while maintaining the temperature between 10° C. to 15° C. The reaction mixture was allowed to warm to room temperature and stirred for 8.0 hours under N₂. The reaction mixture was quenched with water (30 mL) and the organic layer was separated. The aqueous layer was extracted with toluene (20 mL) and the combined organic layer was washed with water (50 mL×2), brine (15%, 30 mL) and dried over sodium sulfate (20 grams). The filtered organic layer was concentrated under vacuum to give perillyl chloroformate as an oil. Weight: 2.5 grams; Yield: 89%. ¹H-NMR (400 MHz, CDCl₃): δ 1.5 (m, 1H), 1.7 (s, 3H), 1.8 (m, 1H), 2.0 (m, 1H), 2.2 (m, 4H), 4.7 (dd, 4H); 5.87 (m, 1H).

Perillyl chloroformate (0.11 grams, 0.55 mmol) was added slowly to a mixture of dimethyl celecoxib (0.2 grams, 0.50 mmol) and potassium carbonate (0.13 grams, 1.0 mmol) in dry acetone (10 mL) over a period of 5 minutes under N₂. The reaction mixture was heated to reflux and maintained for 3 hours. Since TLC analysis indicated the presence of dimethyl celecoxib (>60%), another 1.0 equivalent of perillyl chloroformate was added and refluxed for an additional 5 hours. The reaction mixture was cooled and acetone was concentrated under vacuum to give a residue.

The resulting residue was suspended in water (15 mL) and extracted with ethyl acetate (3×15 mL). The combined organic layer was washed with water (20 mL) followed by brine (15%, 20 mL) and dried over sodium sulfate. The filtered organic layer was concentrated under vacuum to give a residue which was purified by column chromatography [column dimensions: diameter: 1.5 cm, height: 10 cm, silica: 230-400 mesh] and eluted with hexanes (100 mL) followed by a mixture of hexanes/ethyl acetate (95:5, 100 mL). The hexane/ethyl acetate fractions were combined and concentrated under vacuum to give a gummy mass.

The product POH carbamate exhibited a weight of 120 mg and a yield of 31%. ¹H-NMR (400 MHz, CDCl₃): δ 0.9 (m, 2H), 1.4 (m, 2H), 1.7 (m, 7H*), 1.95 (m, 8H*), 2.1 (m, 4H), 2.3 (s, 3H), 4.4 (d, 2H), 4.7 (dd, 2H), 5.6 (br d, 2H), 6.6 (s, 1H), 7.0 (br s, 1H), 7.12 (d, 1H), 7.19 (d, 1H), 7.4 (d, 2H), 7.85 (d, 2H); MS, m/e: 751.8 (M*3%), 574.3 (100%), 530.5 (45%), 396 (6%). *N.B. further 2H overlapping from presumed impurity discounted in NMR integration.

### Example 7—Synthesis of Temozolomide POH Carbamate (3-methyl 4-oxo-3,4-dihydroimidazo[5,1-d][1,2,3,5]tetrazine-8-carbonyl)-carbamic acid-4-isopropenyl cyclohex-1-enylmethyl ester)

The reaction scheme is the following:

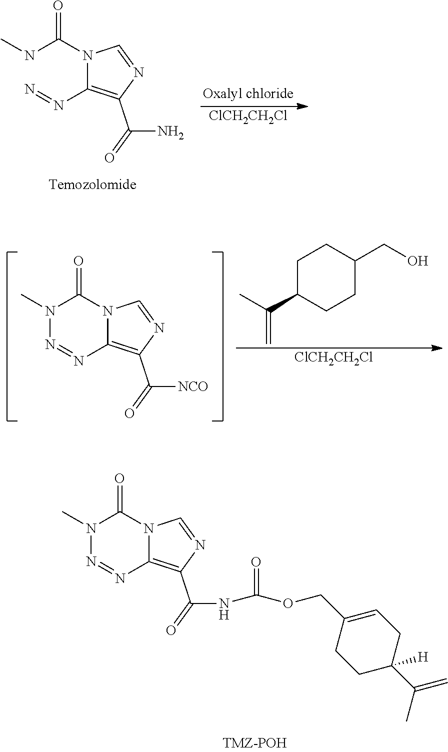

Oxalyl chloride (0.13 grams, 1.0 mmol) was added slowly to a mixture of temozolomide (OChem Incorporation, 0.1 grams, 0.5 mmol) in 1,2-dichloroethane (10 mL) over a period of 2 minutes while maintaining the temperature at 10° C. under N₂. The reaction mixture was allowed to warm to room temperature and then heated to reflux for 3 hours. The excess of oxalyl chloride and 1,2-dichloroethane were removed by concentration under vacuum. The resulting residue was re-dissolved in 1,2-dichlorethane (15 mL) and the reaction mixture was cooled to 10° C. under N₂. A solution of perillyl alcohol (0.086 grams, 0.56 mmol) in 1,2-dichloroethane (3 mL) was added over a period of 5 minutes. The reaction mixture was allowed to warm to room temperature and stirred for 14 hours. 1,2-dichloroethane was concentrated under vacuum to give a residue, which was triturated with hexanes. The resulting yellow solid was filtered and washed with hexanes. Weight: 170 mg; Yield: 89%. ¹H-NMR (400 MHz, CDCl₃): δ 1.4-2.2 (m, 10H), 4.06 (s, 311), 4.6-4.8 (m, 411), 5.88 (br s, 111), 8.42 (s, 111), 9.31 (br s, 111); MS, no molecular ion peak was observed. m/e: 314 (100%), 286.5 (17%), 136 (12%).

Alternatively, temozolomide POH carbamate was synthesized according to the following procedure. Oxalyl chloride (0.13 grams, 1.0 mmol) was added slowly to a mixture of temozolomide (OChem Incorporation, 0.1 grams, 0.5 mmol) in 1,2-dichloroethane (10 mL) over a period of 2 minutes while maintaining the temperature at 10° C. under N₂. The reaction mixture was allowed to warm to room temperature and then heated to reflux for 3 hours. The excess of oxalyl chloride and 1,2-dichloroethane were removed by concentration under vacuum. The resulting residue was re-dissolved in 1,2-dichlorethane (15 mL) and the reaction mixture was cooled to 10° C. under N₂. A solution of perillyl alcohol (0.086 grams, 0.56 mmol) in 1,2-dichloroethane (3 mL) was added over a period of 5 minutes. The reaction mixture was allowed to warm to room temperature and stirred for 14 hours. 1,2-Dichloroethane was concentrated under vacuum to give a residue, which was purified by a short silica-plug column (column dimensions: diameter: 2 cm, height: 3 cm, silica: 230-400 mesh) and eluted with a mixture of hexanes/ethyl acetate (1:1, 100 mL). The hexane/ethyl acetate fractions were combined and concentrated under vacuum to give a white solid residue which was triturated with heptanes and filtered to obtain a white solid. Weight: 170 mg; Yield: 89%. ¹H-NMR (400 MHz, CDCl₃): 1.4-2.2 (m, 10H), 4.06 (s, 3H), 4.6-4.8 (m, 411), 5.88 (br s, 1H), 8.42 (s, 111), 9.31 (br s, 1H); MS, no molecular ion peak was observed, m/e: 314 (100%), 286.5 (17%), 136 (12%).

### Example 8—Synthesis of Rolipram POH Carbamate (4-(3-cyclopentyloxy-4-methoxy

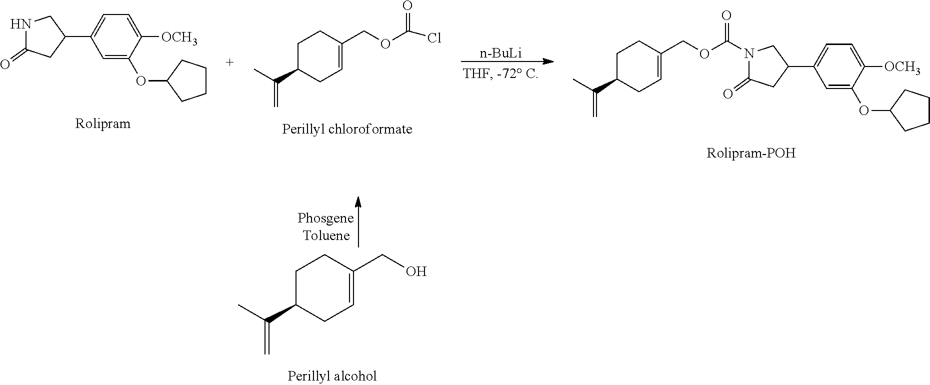

Phosgene (20% in toluene, 13 ml, 26.2 mmol) was added to a mixture of perillyl alcohol (2.0 grams, 13.1 mmol) and potassium carbonate (5.4 grams, 39.1 mmol) in dry toluene (30 mL) over a period of 30 minutes while maintaining the temperature between 10° C. to 15° C. The reaction mixture was allowed to warm to room temperature and stirred for 8.0 hours under N₂. The reaction mixture was quenched with water (30 mL) and the organic layer separated. The aqueous layer was extracted with toluene (20 mL) and the combined organic layer washed with water (50 mL×2), brine (15%, 30 mL) and dried over sodium sulfate (20 grams). The filtered organic layer was concentrated under vacuum to give perillyl chloroformate as an oil. Weight: 2.5 grams; Yield: 89%. ¹H-NMR (400 MHz, CDCl₃): δ 1.5 (m, 1H), 1.7 (s, 3H), 1.8 (m, 1H), 2.0 (m, 1H), 2.2 (m, 4H), 4.7 (dd, 4H); 5.87 (m, 1H).

Butyl lithium (2.5 M, 0.18 mL, 0.45 mmol) was added to a solution of rolipram (GL synthesis, Inc., 0.1 grams, 0.36 mmol) in dry THF at −72° C. over a period of 5 minutes under N₂. After the reaction mixture was stirred for 1.0 hours at −72° C., perillyl chloroformate (dissolved in 4 ml THF) was added over a period of 15 minutes while maintaining the temperature at −72° C. The reaction mixture was stirred for 2.5 hours and quenched with saturated ammonium chloride (5 mL). The reaction mixture was allowed to warm to room temperature and extracted with ethyl acetate (2×15 mL). The combined organic layer was washed with water (15 mL), brine (15%, 15 mL), and then dried over sodium sulfate. The filtered organic layer was concentrated to give an oil which was purified by column chromatography [column dimensions: diameter: 1.5 cm, height: 10 cm, silica: 230-400 mesh] and eluted with a mixture of 8% ethyl acetate/hexanes (100 mL) followed by 12% ethyl acetate/hexanes (100 mL). The 12% ethyl acetate/hexanes fractions were combined and concentrated under vacuum to yield a gummy solid. Weight: 142 mg; Yield: 86%. ¹H-NMR (400 MHz, CDCl₃): δ 1.5 (m, 1H), 1.6 (m, 2H), 1.7 (s, 3H), 1.9 (m, 6H), 2.2 (m, 5H), 2.7 (m, 1H), 2.9 (m, 1H), 3.5 (m, 1H), 3.7 (m, 1H), 3.8 (s, 3H), 4.2 (m, 1H), 4.7 (m, 6H), 5.8 (br s, 1H), 6.8 (m, 3H); MS, m/e: 452.1 (M⁺¹53%), 274.1 (100%), 206.0 (55%).

### Example 9—Treatment of Recurrent Glioblastoma with Perillyl Alcohol

Better treatments for glioblastoma (GBM) patients, in particular in the recurrent setting, are urgently needed. Clinical trials performed in Brazil indicated that intranasal delivery of perillyl alcohol (POH) might be effective in this patient group. NEO100, a highly purified version of POH, was cGMP manufactured to evaluate safety and efficacy of this novel approach in a Phase 1/2a clinical trial in the United States.

This study presents results from a completed Phase 1 study with intranasal NEO100 in recurrent glioblastoma (GBM) patients. NEO100 is a highly pure, cGMP-manufactured version of the natural monoterpene perillyl alcohol. Our results demonstrate that intranasal NEO100 is safe, and can be used to treat recurrent glioblastoma. The historical survival time for first time recurrent GBM for IDH1 wild type is 9.8 months; whereas, the survival time for first time recurrent GBM for IDH1 mutant is 19.32 months. No increase in progression free survival was noted (Mandel J J, Cachia D, Liu D, Wilson C, Aldape K, Fuller G, DeGroot J F: Impact of IDH1 mutation status on outcome in clinical trials for recurrent glioblastoma. J Neurooncol 129:147-154, 2016). Treatment with NEO100 in IDH1 mutant recurrent GBM resulted in unexpected length of progression free survival (average of 32 months) and survival (all still alive, average 32 months) in three patients.

A total of 12 patients with recurrent GBM were enrolled into Phase 1 of this trial. NEO100 was administered by intranasal delivery using a nebulizer and nasal mask. Dosing was four times a day, every day. Four cohorts of 3 patients received the following dosages: 96 mg/dose (384 mg/day), 144 mg/dose (576 mg/day), 192 mg/dose (768 mg/day), and 288 mg/dose (1152 mg/day). Completion of 28 days of treatment was recorded as 1 cycle. Adverse events were documented, and radiographic response via RANO criteria was evaluated every two months. Progression-free and overall survival were determined after 6 and 12 months, respectively (PFS-6, OS-12).

Intranasal NEO100 was well tolerated at all dose levels and no severe adverse events were reported. PFS-6 was 33%, OS-12 was 55%, and median OS was 15 months. Four patients (33%) survived >24 months.

Intranasal glioma therapy with NEO100 was well tolerated. It correlated with improved survival when compared to historical controls, pointing to the possibility that this novel intranasal approach could become useful for the treatment of recurrent GBM.

Prognosis of patients with recurrent glioblastoma remains dismal and better treatment options are urgently needed. Our Phase 1 study evaluated intranasal administration of NEO100, a highly purified version of the natural limonene-related compound perillyl alcohol, as a potential novel treatment for this patient group. Patients with recurrent glioblastoma self-administered NEO100 daily via nebulizer 4 times a day. The safety profile of NEO100 was excellent and there was suggestive evidence of activity. Intranasal NEO100 represents a novel approach to brain cancer therapy and has the potential to become clinically useful to improve treatment outcomes for recurrent glioblastoma patients.

### Introduction

Glioblastoma (GBM, WHO grade IV glioma) is the most common primary malignant brain tumor among adults. Regardless of the treatment regimen, the vast majority of patients relapse and are faced with limited treatment options. The aggressive infiltration of GBM throughout the brain typically limits the efficacy of repeat surgical resection, and tumor cells frequently acquire resistance to further cytotoxic therapy. Therefore, recurrent GBM does not respond well to repeat surgery, re-irradiation and additional rounds of chemotherapy; while these interventions may moderately increase overall survival, the prognosis for these patients remains exceptionally poor. In the U.S. and Canada, the angiogenesis inhibitor bevacizumab has received market approval for the treatment of recurrent GBM.² It is a humanized monoclonal antibody against VEGF (vascular endothelial growth factor) and thus represents a targeted therapy. It can be used alone or in combination with cytotoxic chemotherapy. However, the duration of benefits is short-lived and its impact on overall survival remains limited and unimpressive, which represents a major reason it was not approved by European authorities.³

In view of the persistent medical need for improved treatments, we are investigating a novel type of intervention, intranasal delivery of perillyl alcohol (NEO100), for patients with recurrent GBM. POH (also called p-metha 1,7-diene-6-ol) is a monoterpene isolated from the essential oils of lavender, citrus fruits, peppermint, and several other plants, which synthesize it through the mevalonate pathway.⁴ Extensive preclinical studies provided strong evidence of this natural compound's anticancer potential. The exact mechanism of POH's anticancer effect is unclear, but most likely results from pleiotropic effects that include cell cycle arrest, endoplasmic reticulum stress, and induction of apoptosis.⁵

Because POH was shown to inhibit the enzymatic activity of farnesyl-protein transferase (FPT) of the mevalonate pathway, it was hypothesized that POH might cause inhibition of oncogenic activity of Ras protein, which requires posttranslational farnesylation for plasma membrane anchoring and mitogenic activity.⁶ However, several studies in this context yielded ambiguous results. Most likely, any impact on Ras activity represents only one of several mechanisms by which POH exerts its anticancer effects (see detailed refs.⁵). Despite consistent anticancer activity in a variety of preclinical models, numerous Phase 1 and 2 trials in the late 1990s in patients with different solid tumors were unable to demonstrate convincing therapeutic activity. In these studies, POH was formulated in gelatin capsules and given orally in rather large doses of several grams 3-4 times daily. Gastrointestinal toxicity proved dose-limiting, and some patients quit the trials due to unrelenting, chronic malaise (fatigue, nausea, belching, reflux, diarrhea or constipation).^{7, 9} As a result, oral POH was abandoned and did not enter clinical practice.

Nasal delivery of chemotherapy is envisioned as a novel, paradigm-shifting platform to deliver therapeutics to the brain, while minimizing systemic toxicity and first-pass metabolism.^{10, 12} Effective nose-to-brain delivery has been demonstrated in a variety of non-cancer conditions, such as migraine, stroke, and other neurological conditions.^{9, 13} For example, intranasal insulin was shown to improve cognition in early Alzheimer's disease.^{14, 15} Although not yet fully characterized, the presumed mechanism of brain drug uptake is thought to involve the olfactory and trigeminal nerves, and the nasal mucosa. Combined, these elements facilitate direct access and quick absorption of drugs, thereby providing for greater bioavailability and rapid onset of drug responses.^{13 16 17} However, despite these distinct benefits, nasal delivery of cancer therapeutics is not established in clinical practice.

Phase 2 studies in Brazil, undertaken with recurrent malignant glioma patients, pioneered intranasal delivery of POH as a novel paradigm of cancer therapy. Commercial-grade POH was self-administered four times daily. Several reports published from these studies indicated that this alternative mode of drug delivery harbors the potential to achieve activity in this patient group.^{18, 20} As well, there was good tolerance, without long-term CNS or systemic severe adverse events, and patient compliance reportedly was very high (>95%).²⁰ Radiographic regression was reported.^{19, 20}

We set out to investigate the clinical safety and activity of intranasal NEO100, a highly purified form of POH produced under current good manufacturing practice (cGMP) conditions, in patients with recurrent GBM. A Phase 1/2a trial is ongoing, and here we are reporting the results from the completed Phase 1 part. 2. Patients and Methods

Phase 1 Trial The ongoing interventional clinical trial entitled “An Open-Label, Phase 1/2A Dose Escalation Study of Safety and Efficacy of NEO100 in Recurrent Grade IV Glioma” [ClinicalTrials.gov Identifier: NCT02704858] is a multi-center study. Participant institutions are Cleveland Clinic, University of Washington/Seattle, University of Wisconsin, and the University of Southern California. It is sponsored by NeOnc Technologies, Inc. (Los Angeles, CA) with ClinDatrix, Inc. (Irvine, CA), as the Clinical Data Management CRO. The patients were enrolled under institutional review board (IRB)-approved protocols and after signing appropriate IRS-approved informed consent forms. For the Phase 1 portion of this trial, the first patient was enrolled in April of 2017, and the 12^{th} patient entered in June of 2019. The primary objectives of Phase 1 were: (i) to determine the safety and tolerability of intranasal administration of NEO100, and (ii) to identify the maximum tolerated dose of NEO100.

NEO100 Administration—NEO100 is highly purified perillyl alcohol that was manufactured under cGMP conditions at Norac Pharma (Azusa, CA). It is delivered four times a day by intranasal administration using a nebulizer and nasal mask. After initial demonstration and instructions by a nurse in the clinic, patients self-administer each dose. NEO100 is provided to each patient formulated as a 10% stock solution in ethanol:glycerol (50:50, v/v). Prior to each use, the stock solution is diluted with water and filled into the nebulizer.

Main Inclusion Criteria—Among the inclusion criteria are the following. (i) Radiographically confirmed progression or recurrent grade IV glioma, and on a stable dose of steroid for at least 5 days. (ii) Patients must have failed previous radiation and temozolomide treatment. (iii) Age 18 years. (iv) ECOG performance status of 0-2, or KPS 60. (v) An expected survival of at least 3 months. (vi) Baseline MRI with gadolinium within two weeks of entry into the trial. (vii) Seizures controlled on a stable dose of anti-epileptics for two weeks prior to enrollment.

Response Assessment—Patients undergo gadolinium-enhanced brain MRI as part of standard care. Baseline tumor measurement is performed within 2 weeks of registration and assessed by RANO criteria (Response Assessment in Neuro-Oncology). MR1s are repeated after every even 28-day cycle (i.e., cycles 2, 4, 6) and whenever disease progression is suspected based on clinical symptoms. Tumor response is assessed using both the MacDonald and the RANO response criteria for high-grade gliomas, which considers radiologic imaging, neurological status and steroid dosing. Safety is evaluated throughout the trial by the incidence of adverse events (AEs), physical examination findings, vital signs and clinical laboratory test results. AEs are graded for severity using NCI Common Terminology Criteria for Adverse Events v.4.0.²¹

### Results

Presented here are results from the completed Phase 1 part of an ongoing Phase 1/2a study of intranasally administered NEO100 in patients with recurrent GBM after failure of standard chemoradiation with temozolomide. Twelve patients were enrolled (demographics and baseline characteristics are shown in Table 1). Successive cohorts of 3 patients each received intranasal NEO100 at escalating dosages of 384 mg/d, 576 mg/d, 768 mg/d and 1152 mg/d. Patients self-administered these amounts, which were divided into 4 equal doses approximately 5-6 hours apart throughout each day.

No severe (grade 3 or 4) adverse effects were noted in any of the cohorts during any of the monthly cycles. Other adverse effects (grade 1) consisted of nasal soreness or itching, runny nose, skin irritation around the nose, or headache. Repeated grade 2 leukopenia was noted in one patient of Cohort 2, but causality to NEO100 treatment was unclear (Table 2).

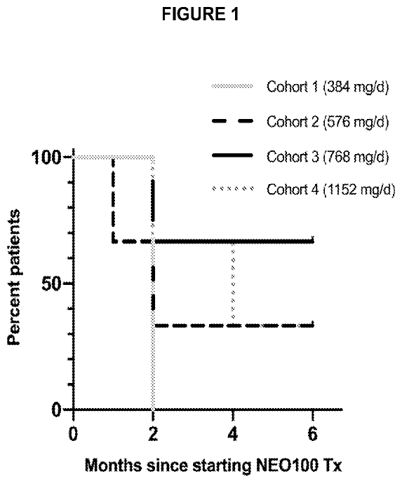

Initially, NEO100 treatment was scheduled for a continuous 6-month treatment. Patients who had stable disease at 6 months were allowed to continue treatment on an extended use protocol, whereas patients who progressed early discontinued the treatment. Progression-free survival during the first 6 months is summarized in FIG. 1 and Table 3. As shown, patients in Cohort 1 (lowest dose) only completed 2 cycles (i.e., 2 months) of NEO100 treatment, due to progressive disease at the end of these cycles. In Cohort 2, two patients also experience progressive disease early on (after 1 and 2 cycles), while the third patient (ID 202) had stable disease at 6 months and since then has continued to administer NEO100 for a total of 33 cycles at this time. Her tumor has shrunk by greater than 75% as measured via MRI. In Cohort 3, only 1 patient terminated treatment early due to progressive disease, whereas the other two patients were stable at 6 months and therefore continued treatment. One of these two patients (ID 302) completed 11 cycles, followed by another 16 months without NEO100 treatment, and is still alive. The other (ID 301) has been continuing treatment for a total of 24 cycles and is still alive. This patient also had a complete radiographic remission, which has continued to this date. In Cohort 4, two patients did not complete the full 6-month treatment due to progression at 2 and 4 months, respectively. One of these patients (ID 402) survived for another 13 months after discontinuation of NEO100. Another (ID 401) was lost to follow-up right after completion of 4 cycles and his current status is unknown. The third patient in this cohort (ID 403) presented with stable disease at 6 months, but thereafter rapidly worsened and died 3 months later. In all, PFS-6 was 33% among the entire group of patients (n=12) enrolled in this Phase 1, with Cohort 1 having the lowest (0%) and Cohort 3 having the highest (67%) PFS-6 (FIG. 1).

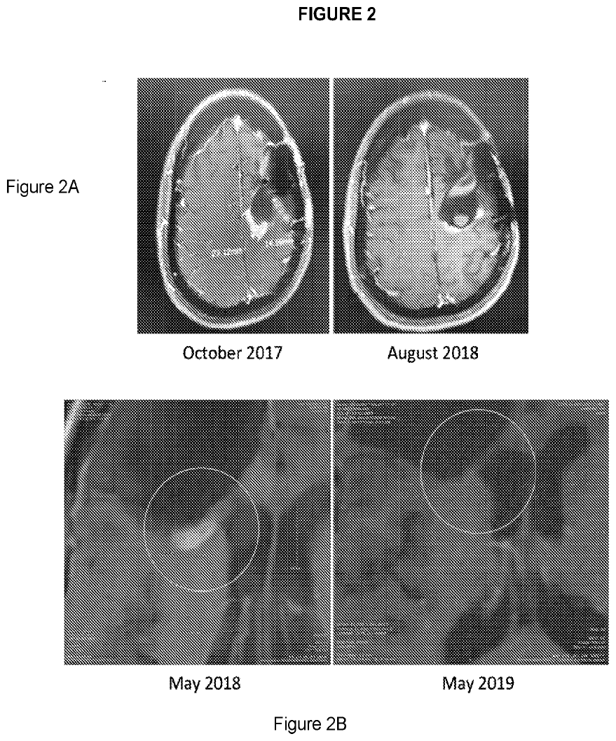

Examples of radiographic responses are presented in FIG. 2, showing a partial response after 10 months and a complete response after 12 months of NEO100 treatment. Overall survival at 12 months (OS-12) was 55%, at 24 months (OS-24) it was 37%, and median OS was 15 months

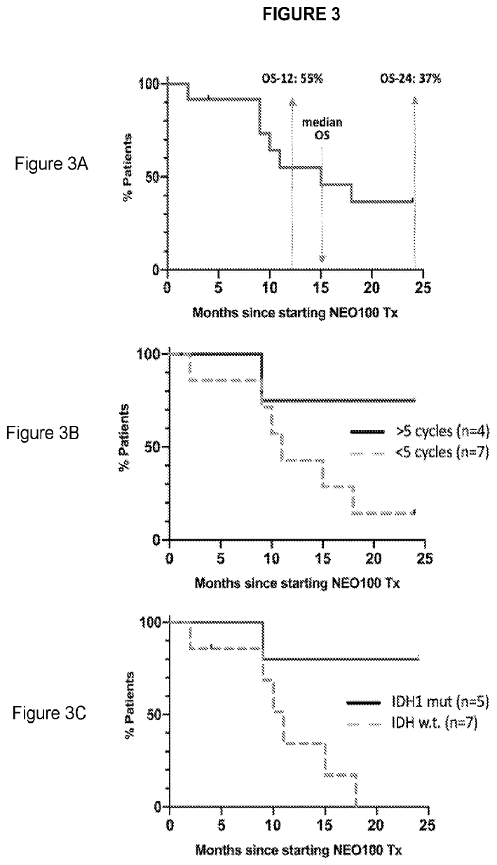

(FIG. 3A). In all, there were several patients with notably long survival: four patients survived at least 24 months, and three of these are still alive (Table 3). Thus, despite only 33% PFS-6, median OS of 15 months emerged as an encouraging result. For further analysis, we separated all patients into two groups: those that had completed at least 6 cycles (n=4) of NEO100, and those that had not (n=?). The latter group included one patient with 1 cycle, 6 patients with 2 cycles, and 1 patient with 4 cycles (who was lost to follow-up immediately after completing 4 cycles, and therefore was omitted from the comparison).

Intriguingly, there was a noticeable difference in longer-term survival between these two groups, although it did not reach statistical significance. As shown in FIG. 3B, for those 4 patients who completed at least 6 cycles, OS-24 was 75%. In comparison, for the evaluable 6 patients who completed only 1 or 2 cycles, OS-24 was 14%. However, despite the poorer outcome of this second group as compared to the first group, median OS was a notable 11 months, again demonstrating that despite early progression the longer-term survival was quite encouraging.

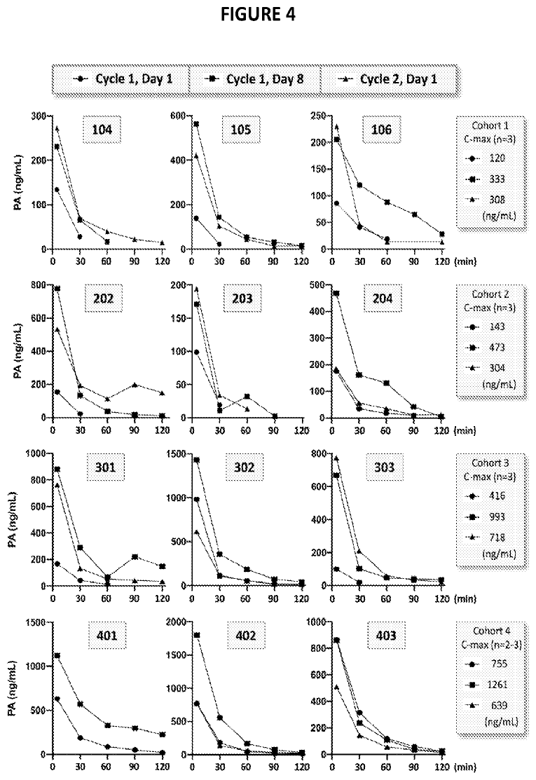

We also analyzed overall survival based on the status of the isocitrate dehydrogenase 1 (IDH1) gene. Mutations in amino acid 132 of IDH1 are present in more than 70% of grade II and III astrocytomas and olgiodendrogliomas as well as glioblastomas that develop from these lesions. See N. England J. Med. 2009; 360:765-773. IDH1/IDH2 mutation analysis can be done as part of a standard clinical laboratory testing protocol using SNaPshot Multiplex PCR (polymerase chain reaction) See, e.g., http://www.labcorp.com/test/481484/i-dh1-idh2-i-mutation-analysis, retreived Nov. 11, 2021, https://www.mayocliniclabs.com/test-catalog/Clinical+and+Interpretive/92361, retrieved Nov. 11, 2021, https://www.mdanderson.org/research/research-resources/core-facilities/molecular-diagnostics-lab/services/idh1-mutation-analysis.html, retrieved Nov. 11, 2021. Mutations in this gene are known to confer a survival advantage for newly diagnosed glioma patients.²² As shown in FIG. 3C, there was significantly longer (p=0.018) overall survival for patients with IDH1 mutant tumors, with 4 of 5 patients (80%) surviving at least 24 months. In comparison, none of the patients with wild type IDH1 survived beyond 18 months, although median OS still was a notable 11 months. The presence of perillic acid (PA) was determined in plasma obtained from all patients at different time points after administration of the first daily dose of intranasal NEO100. These blood draws were done on Day 1 and 8 of the first 28-day cycle, and repeated on the first day of the second cycle. PA is a major metabolite of perillyl alcohol and is more stable, making it a convenient, easy to detect marker of POH exposure. As shown in FIG. 4, plasma concentrations of PA were readily quantifiable and present at maximum concentrations at 5 minutes after NEO100 administration, with an initial half-life of approximately 20 minutes. Maximum plasma PA concentrations on average were higher in patients administering the higher dosages. As well, within each cohort, these concentrations were noticeable higher during the two later days, as compared to the measurements from the very first dose administration (Day 1 of Cycle 1). Despite noticeable interpatient variability in absolute values, Cmax was reduced by >90% in most patients within 2 hours after intranasal delivery. In all, these data indicated rapid drug entry into the systemic circulation that was followed by first-order kinetics of elimination and lack of accumulation.

### Discussion

The present study provides evidence that intranasal NEO100, when delivered four times a day, is safe and potentially effective in recurrent GBM patients. The treatment was very well tolerated at all dose levels and no severe adverse events were reported. At the highest dosage used, 1152 mg/day divided into 4 equal doses of 288 mg, MTD was not reached. These results are consistent with those obtained in Phase 1/2 studies in Brazil that used commercial-grade POH in patients with recurrent GBM, grade Ill anaplastic astrocytoma and anaplastic oligodendroglioma, although at lower dosages of 133 mg qid (534 mg/day).^{18, 20} In those studies, adherence to the protocol was high (>95%) and occasionally caused nose soreness but no severe adverse effects, even after several years of continuous application.²

Despite the small patient number in our current study, initial analysis of efficacy of intranasal NEO100 for recurrent GBM patients appears promising. PFS-6 was 33%, OS-6 was 92%, OS-12 was 58%, and four patients (33%) survived >24 months. This compares very favorably to prior single-agent studies with recurrent GBM patients, several of which are summarized in Supplementary Table 1. For instance, Wong et al. reviewed eight Phase 2 studies with various treatments performed during the pre-temozolomide era, which averaged 21% OS-12 and 5.7 months median OS.²³ Several newer studies, completed over the past 8 eight years mostly with patients that had failed standard chemoradiation with temozolomide (a.k.a. the Stupp protocol 4 yielded mixed results and achieved only incremental improvements in survival. For example, alternating electric fields (tumor treatment fields, TTFs, NovoTTF-100A) emerged as a conceptually novel approach a decade ago, but it did not show improved outcomes in the recurrent setting²⁵ as compared to historical controls or conventional chemotherapy, such as lomustine²⁶ or fotemustine.²⁷ Bevacizumab was granted accelerated approval for the treatment of recurrent GBM in the U.S., although its impact on OS-12 and median OS remained muted.^{28, 30}

A very recent trial with nivolumab, a fully human monoclonal antibody targeting the programmed death-1 (PD-1) immune checkpoint receptor, also did not yield substantial improvements, and survival results were comparable to those achieved with conventional chemotherapy or bevacizumab.³¹

Two very recent trials reported outcomes that pushed the median OS beyond the 1-year mark (Supplementary Table 1). One study used Toca-511 (vocimagene amiretrorepvec), a nonlytic retroviral replicating vector that delivers yeast cytosine deaminase, which converts separately administered Toca FC (extended-release 5-fluorocytosine) into the antimetabolite 5-fluorouracil.³² This trial achieved an OS-12 of 55% and median OS of 13.6 months. Similar results were obtained with direct intratumoral delivery of PVSRIPO, a recombinant polio-rhinovirus chimera that recognizes the poliovirus receptor CD155, which is commonly expressed on the surface of tumor cells.³³ This trial achieved an OS-12 of 54% and median OS of 12.5 months. Results from our current study on intranasal NEO100 compare very favorably to these improved outcomes, as we achieved an OS-12 of 55% and median OS of 15 months.

An important advantage of our study lies in its very low toxicity, non-invasiveness, and lack of serious adverse events, emphasizing that this treatment approach does not lead to deterioration of quality of life for the patients. In comparison, many other treatments mentioned above have less than optimal safety profile. For example, nitrosoureas are known for their bone marrow suppression, liver/renal toxicity, or interstitial lung disease, and bevacizumab may cause hemorrhage and hypertension. Direct administration via convection-enhanced delivery, as is practiced in case of PVSRIPO, is invasive and includes all risks associated with surgical catheter placement and removal. In general, combination regimens do not produce evidence for superior activity, but commonly produce more toxicity.

We further made the intriguing observation that even those patients who progressed before completion of the planned 6 months of treatment with NEO100 lived longer than expected. Upon progression, these patients were switched to a mixture of best standard of care as per their neurooncologist. Moreover, there may have been pseudoprogression on MRI scan, leading to premature stoppage of NEO100. It will be important to pay particular attention to these unresolved issues in the Phase Ila part of this study. Another intriguing result was our observation that patients with IDH1 mutation appeared to have a survival advantage. IDH1 gene mutation is a known predictor of better overall survival in malignant glioma.²² However, while this link has been firmly established in the case of newly-diagnosed patients, it is not clear whether it also applies to the recurrent setting, as inconsistent outcomes (on small numbers of patients) have been reported. For instance, Mandel et al.³⁵ reported that the IHD1 mutation might have a positive influence on survival, although only at first recurrence. However, another report by Tabei et al.³⁶ was unable to confirm a positive correlation of IDH1 mutation and survival after first progression. Our results with NEO100-treated patients do show that those with IDH1 mutant status survived significantly longer from the time of enrollment in this trial.

In conclusion, intranasal glioma therapy with NEO100 was well tolerated. It correlated with improved survival when compared to historical controls, pointing to the possibility that this novel conceptual approach could become useful for the treatment of recurrent GBM. Due to its very low toxicity profile, it might offer the possibility of combining this regimen with other, more taxing approaches without increasing adverse events. As well, based on the facile administration process and continued quality of life, patients who progress on intranasal NEO100 might be more inclined to pursue further lines of therapy. Although resistance mechanisms against NEO100 have not yet been identified and characterized, one might surmise that standard postprogression treatments and approaches presented in Supplementary Table 1 could still unfold significant activity and benefit for such patients.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art, that changes and modifications may be made without departing from the spirit and scope of the invention. The matter set forth in the foregoing description and accompanying drawings is offered by way of illustration only and not as a limitation.

### REFERENCES

1. Mehta M, Wen P, Nishikawa R, Reardon D, Peters K. Critical review of the addition of tumor treating fields (TTFields) to the existing standard of care for newly diagnosed glioblastoma patients. Crit Rev Oncol Hemaol. 2017; 111:60-65.
2. Ghiaseddin A, Peters K B. Use of bevacizumab in recurrent glioblastoma. CNS Oncol. 2015; 4(3):157-169.
3. de Lemos M L, Markarian A, Chan E. Schaff K. Walisser S. Clinical ef-fectiveness of bevacizurnab in patients with recurrent brain tumours: a population-based evaluation. J Oncol Pharm Pract. 2018; 24(1):33-36,
4. Crowell P L, Elson C E. Isoprenoids, health and disease. In: Wildman R E C, ed. Nutraceuticals and Functional Foods. Boca Raton, FL: CRC Press; 2001:31-54.
5. Chen T C, Fonseca C O, Sch6nthal A H. Preclinical development and clinical use of perillyl alcohol for chemoprevention and cancer therapy. Am J Cancer Res. 2015; 5(5):1580-1593.
6. Gelb M H, Tamanoi F, Yokoyama K, Ghomashchi F, Esson K, Gould M N. The inhibition of protein prenyltransferases by oxygenated metabolites of limonene and perillyl alcohol. Cancer Lett. 1995; 91(2):169-175.
7. Bailey H H, Levy D, Hari s L S, et al. A phase 11 trial of daily perillyl alcohol in patients with advanced ovarian cancer: Eastern Cooperative Oncology Group Study E2E96. Gynecol Oncol. 2002; 85(3):464-468.
8. Liu G, Oettel K, Bailey H, et al. Phase II trial of perillyl alcohol (NSC 641066) administered daily in patients with meta-static androgen independent prostate cancer. Invest New Drugs. 2003; 21(3):367-372.
9. Meadows S M, Mulkerin D, Berlin J, et al. Phase II trial of perillyl alcohol in patients with metastatic colorectal cancer. Int J Gastrointest Cancer. 2002; 32 (2-3):125-128.
10. Bitter C. Suter-Zimmermann K, Surber C. Nasal drug delivery in humans. Curr Probl Dermatol. 2011; 40:20-35.
11. Dhuria S V, Hanson L R, Frey W H 11. Intranasal delivery to the central nervous system: mechanisms and experimental considerations. J Pharm Sci. 2010; 99(4):1654-1673.
12. Peterson A. Bansal A, Hofman F. Chen T C, Zada G. A systematic review of inhaled intranasal therapy for central nervous system neoplasms: an emerging therapeutic option. J Neurooncol. 2014; 116(3):437-446.
13. Gizurarson S. Anatomical and histological factors affecting intranasal drug and vaccine delivery. Curr Drug Deliv. 2012; 9(6):566-582.
14. Chapman C D, Schi\check{o}th H B, Grillo C A, Benedict C. Intranasal insulin in Alzheimer's disease: food for thought. Neuropharmacology. 208; 136 (Pt B): 196-201.
15. Reger M A, Watson G S, Green P S, et al. Intranasal insulin improves cognition and modulates beta-amyloid in early AD. Neurology. 2008; 70(6):440-448.
16. Ilium L. Nasal drug delivery—recent developments and future prospects. J Control Release. 2012; 161(2):254-263.
17. Ugwoke M I, Agu R U, Verbeke N. Kinget R. Nasal mucoadhesive drug delivery: background, applications, trends and future perspectives. Adv Drug Deliv Rev. 2005; 57(11):1640-1665.
18. da Fonseca C O, Schwartsmann G3, Fischer J, et al. Preliminary re-sults from a phase I/II study of perillyl alcohol intranasal admin-istration in adults with recurrent malignant gliomas. Surg Neurol. 2008; 70(3):259-266.
19. da Fonseca C O, Simão M, Lins I R, Caetano R O. Futuro D, Quirico-Santos T. Efficacy of monoterpene perillyl alcohol upon survival rate of patients with recurrent glioblastoma. J Cancer Res Clin Oncol. 2011; 137(2):287-293.
20. da Fonseca C O, Teixeira R M, Silva J C, et al. Long-term outcome in pa-tients with recurrent malignant glioma treated with perillyl alcohol inhalation. Anticancer Res. 2013; 33(12):5625-5631.
21. National Cancer Institute. Common terminology criteria for adverse events (CTCAE) Version 4.0. 2009. Accessed Aug. 30. 2017.
22. Huang L E. Friend or foe-IDH1 mutations in glioma 10 years on. Carcinogenesis. 2019; 40(11):1299-1307.
23. Wong E T. Hess K R, Gleason M J, et al. Outcomes and prognostic factors in recurrent glioma patients enrolled onto phase 11 clinical trials. J Clin Oncol. 1999; 17(8):2572-2578.
24. Stupp R, Mason W P, van den Bent M J, et al.; Radiotherapy plus concomitant and adjuvant temozolomide for glioblas-toma. N Engl J Xed. 2005; 352(10):987-996.
25. Stupp R, Wong E T, Kanner A A, et al. NovoTTF-100A versus physician's choice chemotherapy in recurrent glioblastoma: a randomised phase III trial of a novel treatment modality. Eur J Cancer. 2012; 48(14):2192-2202.
26. Batchelor T T, Mulholland P, Neyns B, et al. Phase III randomized trial comparing the efficacy of cediranib as monotherapy, and in combination with lomustine, versus lomustine alone in patients with recurrent glioblastoma. J Clin Oncol. 2013; 31(26):3212-3218.
27. Brandes A A Finocchiaro G, Zagonel V, et al. AVAREG: a phase II, randomized, noncomparative study of fotenmustine or bevacizumab for patients with recurrent glioblastoma. Neuro Oncol. 2016; 18(9):1304-1312.
28. Field K M, Simes J, Nowak A K, et al.; CABARET/COGNO investigators. Randomized phase 2 study of carboplatin and bevacizumab in recurrent glioblastoma. Neuro Oncol. 2015; 17(11):1504-151.3,
29. Heiland D H, Masalha W, Franco P, Machein M R, Weyerbrock A. Progression-free and overall survival in patients with recurrent glioblastoma multiforme treated with last-line bevacizumab versus bevacizumab/lomustine. J Neurooncol. 2016:126(3):567-575.
30. Taal W, Oosterkamp H M, Walenkamp A M. et al. Single-agent bevacizumab or lomustine versus a combination of bevacizumab plus lomustine in patients with recurrent glioblastoma (BELOB trial): a randomised controlled phase 2 trial. Lancet Oncol. 2014; 15(9):943-953.
31. Reardon D A, Brandes A A, Omuro A, et al. Effect of Nivolumab vs bevacizumab in patients with recurrent glioblastoma: The CheckMate 143 Phase 3 randomized clinical trial. JAMA Oncol. 2020.
32. Cloughesy T F, Landolfi J, Hogan D J, et al. Phase 1 trial of vocimagene amiretrorepvec and 5-fluorocytosine for recurrent high-grade glioma. Sci Transl Med. 2016; 8(341):341ra375.
33. Desjardins A, Gromeier M, Herndon J E II, et al. Recurrent glioblastoma treated with recombinant poliovirus. N Engl J Med, 2018; 379(2):150-161.
34. Weller M, Cloughesy T, Perry J R, Wick W. Standards of care for treatment of recurrent glioblastoma—are we there yet? Neuro Oncol. 2013; 15(1):4-27.
35. Mandel J J, Cachia D, Liu D, et al. Impact of IDH1 mutation status on outcome in clinical trials for recurrent glioblastoma. J Neurooncol. 2016; 129(1):147-154.
36. Tabei Y, Kobayashi K, Saito K. et al. Survival in patients with glioblastoma at a first progression does not correlate with isocitrate dehydrogenase (IDH)1 gene mutation status. Jpn J Clin Oncol. 2020 Sep. 5; doi: 10.1093/jjco/hyaa162; online ahead of print.

TABLE 1
Patient demographics and baseline characteristics

| Patient | | Age | Ethnic | MGMT | IDH1 | | |
|---|---|---|---|---|---|---|---|
| ID | Gender | (years) | group | status | status | KPS | Tumor Location |
| 104 | M | 70 | Caucasian | unmethylated | wild type | 90 | left occipital |
| 105 | M | 62 | Asian | methylated | mutated | 90 | left temporal |
| 106 | F | 51 | Caucasian | methylated | mutated | 90 | right frontal |
| 202 | F | 44 | Hispanic | unmethylated | mutated | 80 | fan frontal |
| 203 | F | 58 | Caucasian | unmethylated | wild type | 80 | left frontal |
| 204 | M | 54 | Caucasian | unmethylated | wild type | 90 | left superior temporal |
| 301 | F | 39 | Caucasian | unknown | mutated | 90 | right frontel |
| 202 | F | 62 | Asian | unknown | mutated | 90 | left parietal |
| 303 | F | 42 | Hispanic | unmethylated | wild type | 70 | midline |
| 401 | M | 69 | Caucasian | unmethylated | wild type | 80 | right temporal |
| 402 | F | 53 | Hispanic | methylated | wild type | 90 | right parietal |
| 403 | M | 70 | Hispanic | methylated | wild type | 90 | right parietal |

TABLE 2
Adverse events attributable to NEO100 administration

| Number of events, | NEO100 dose level (mg/day) | | | | |
|---|---|---|---|---|---|
| according to body system and grade | 384 | 576 | 768 | 1152 | Causality |
| General disorder or administration-site condition: | | | | | |
| Fatigue, grade 1 | 1 | — | — | — | possibly related |
| Nervous system disorder: | | | | | |
| Headache, grade 1 | 1 | — | — | — | probably related |
| Skin and subcutaneous tissue disorders: | | | | | |
| Piloerection, grade 1 | 1 | — | — | — | possibly related |
| Skin irritation around nose, grade 1 | — | — | 1 | — | definitely related |
| Respiratory, thoracic and mediastinal disorders: | | | | | |
| Rhinorrhea, grade 1 | 2 | — | — | 1 | definitely related |
| Nasal dryness, grade 1 | 1 | — | 1 | — | probably related |
| Nasal prurilus, grade 1 | 1 | — | — | — | probably related |
| Nasal discomfort, grade 1 | 1 | 1 | — | — | probably related |
| Cough, grade 1 | — | — | — | 1 | definitely related |
| Blood and lymphatic system disorders: | | | | | |
| Leukopenia, grade 2 | — | 2 | — | — | possibly related |
| Total no. of patients with an event: | 3 | 2 | 1 | 1 | |

TABLE 3
Cohorts, dosages and results

| Patient | | Dosage | Completed | RANO | Survival since start of | Current | NEO100 Tx |
|---|---|---|---|---|---|---|---|
| ID | Cohort | (mg/day) | cycles* | ** | NEO100 Tx (months) | status | ongoing |
| 104 | 1 | 384 | 2 | PD | 18 | deceased | N/A |
| 105 | 1 | 384 | 2 | PD | 9 | deceased | N/A |
| 106 | 1 | 384 | 2 | PD | 33 | deceased | N/A |
| 202 | 2 | 576 | 33 | SD | 33 | alive | yes |
| 203 | 2 | 576 | 2 | PD | 11 | deceased | N/A |
| 204 | 2 | 576 | 1 | N/A | 2 | deccasad | N/A |
| 301 | 3 | 768 | 24 | SD | 24 | alive | yes |
| 302 | 3 | 768 | 11 | SD | 27 | alive | no |
| 303 | 3 | 768 | 2 | PD | 10 | deceased | N/A |
| 401 | 4 | 1152 | 4 | PD | >4 | unknown | no |
| 402 | 4 | 1152 | 2 | PD | 15 | deceased | N/A |
| 403 | 4 | 1152 | 8 | SD | 9 | deceased | N/A |

*each cycle is 28 days
** performed at end of even-numbered cycles and at 6 month final

SUPPLEMENTARY TABLE 1

| | | | OS-6 | OS-9 | OS-12 | median OS |
|---|---|---|---|---|---|---|
| Study | Year | Treatment | (%) | (%) | (%) | (months) |
| Wong et al. | 1999 | various | | | 21 | 5.7 |
| Stupp et al. | 2012 | NovoTTF-100A | 53 | 33 | 20 | 6.6 |
| Batchelor et al. | 2013 | Lomustine | 70 | 52 | 41 | 9.8 |
| Taal et al. (BELOB) | 2014 | Bevacizumab | 64 | 38 | 26 | 8.0 |
| Field et al. (CABARET) | 2015 | | 61 | 39 | 24 | 7.5 |
| Heiland et al. | 2016 | | 18 | 12 | 10 | 4.1 |
| Brandes et al. (AVAREG) | 2016 | Fotemustine | 73 | 47 | 40 | 8.7 |
| Cloughesy et al. | 2016 | Toca-511 | 96 | 84 | 55 | 13.6 |
| Desjardins et al. | 2018 | PVSRIPO | 90 | 71 | 54 | 12.5 |
| Reardon et al. (CheckMate 143) | 2020 | Nivolumab | 72 | 52 | 42 | 9.8 |
| Current Study | 2020 | NEO100 | 92 | 73 | 55 | 15.0 |

What is claimed is:

1. A method of treating a tumor of the nervous system in a patient, wherein the patient has a mutated isocitrate dehydrogenase 1 (IDH1) gene, the method comprising administering to the patient a pharmaceutical composition comprising perillyl alcohol (POH) or a perillyl alcohol carbamate, wherein the perillyl alcohol carbamate is perillyl alcohol covalently bound via a carbamate linking group to a therapeutic agent.

2. The method of claim 1, wherein the tumor of the nervous system is glioblastoma.

3. The method of claim 2, wherein the glioblastoma is recurrent glioblastoma.

4. The method of claim 1, wherein the therapeutic agent is a chemotherapeutic agent.

5. The method of claim 4, wherein the chemotherapeutic agent is selected from the group consisting of a DNA alkylating agent, a topoisomerase inhibitor, an endoplasmic reticulum stress inducing agent, a platinum compound, an antimetabolite, an enzyme inhibitor, a receptor antagonist, a therapeutic antibody, and combinations thereof.

6. The method of claim 4, wherein the chemotherapeutic agent is dimethyl-celecoxib (DMC), irinotecan (CPT-11), temozolomide or rolipram.

7. The pharmaceutical composition of claim 1, wherein the pharmaceutical composition is administered by inhalation, intranasally, orally, intravenously, subcutaneously or intramuscularly.

8. The method of claim 1, wherein the pharmaceutical composition is administered using a nasal delivery device.

9. The method of claim 8, wherein the nasal delivery device is selected from the group consisting of an intranasal inhaler, an intranasal spray device, an atomizer, a nebulizer, a metered dose inhaler (MDI), a pressurized dose inhaler, an insufflator, a unit dose container, a pump, a dropper, a squeeze bottle and a bi-directional device.

10. The method of claim 1, further comprising treating the patient with radiation.

11. The method of claim 1, further comprising administering to the patient a chemotherapeutic agent.

12. The method of claim 1, wherein the perillyl alcohol carbamate is selected from the group consisting of perillyl alcohol conjugated with Dimethyl Celecoxib, Temozolomide POH Carbamate (3-methyl 4-oxo-3,4-dihydroimidazo[5,1-d][1,2,3,5]tetrazine-8-carbonyl)-carbamic acid-4-isopropenyl cyclohex-1-enylmethyl ester) and Rolipram POH Carbamate (4-(3-cyclopentyloxy-4-methoxy phenyl)-2-oxo-pyrrolidine-1-carboxylic acid 4-isopropenyl cyclohex-1-enylmethyl ester) or mixtures thereof.

13. The method of claim 1, wherein the (S)-perillyl alcohol has a purity greater than about 99.0% (w/w), or greater than about 99.5% (w/w).

14. The method of claim 13, wherein the (S)-perillyl alcohol has a purity greater than about 99.0% (w/w).

15. The method of claim 13, wherein the (S)-perillyl alcohol has a purity greater than about 99.5% (w/w).
